(19) United States
(12) Patent Application Publication
(10) Pub. No.: US 2024/0042029 A1
(43) Pub. Date: Feb. 8, 2024

(54) DELIVERY OF MOLECULES TO CELLS USING TROGOCYTOSIS AND ENGINEERED CELLS

(71) Applicant: REGENTS OF THE UNIVERSITY OF MINNESOTA, MINNEAPOLIS, MN (US)

(72) Inventors: Branden Moriarity, Minneapolis, MN (US); Beau Webber, Minneapolis, MN (US); Kenta Yamamoto, Minneapolis, MN (US); Joseph Skeate, Minneapolis, MN (US)

(73) Assignee: REGENTS OF THE UNIVERSITY OF MINNESOTA, MINNEAPOLIS, MN (US)

(21) Appl. No.: 18/266,220

(22) PCT Filed: Dec. 8, 2021
(86) PCT No.: PCT/US2021/062420

§ 371 Date: Jun. 8, 2023

Related U.S. Application Data
(60) Provisional application No. 63/122,590, filed on Dec. 8, 2020.

(51) Int. Cl. A61K 39/00; C12N 5/0783; C12N 15/90; C12N 15/11; C12N 9/22

(52) U.S. Cl. CPC A61K 39/4631; C12N 5/0646; C12N 15/907; C12N 15/11; C12N 9/22; A61K 39/4611; A61K 39/4613; C12N 2502/99; C12N 2310/20; C12N 2800/80

(57) ABSTRACT

Provided herein are methods and compositions for targeted delivery of cargo molecules, including, for example, gene editing reagents, RNA binding proteins, therapeutic agents, or other cargo via trogocytosis. In particular, provided herein are genetically modified donor cells as well as methods of using such genetically modified donor cells to deliver cargo of interest fused to a transmembrane receptor to a specific acceptor target cell.

### CROSS-REFERENCE TO RELATED APPLICATIONS

This application claims priority to U.S. Provisional Application No. 63/122,590 filed on Dec. 8, 2020, the contents of which are incorporated by reference in its entirety.

### SEQUENCE LISTING STATEMENT

A Sequence Listing accompanies this application and is submitted as an ASCII text file of the sequence listing named “920171_00446_ST25.txt” which is 35,095 bytes in size and was created on Dec. 8, 2021. The sequence listing is electronically submitted via EFS-Web with the application and is incorporated herein by reference in its entirety.

### BACKGROUND

The ability to introduce specific sequence changes to a cell's DNA holds great promise for the treatment of human disease. This includes conditions driven by known genetic mutations and viral infections, in addition to more complex diseases. The therapeutic potential of this approach is contingent on efficient delivery of gene editing reagents to target cells and tissues. This can be accomplished by either direct in vivo delivery of constructs encoding gene editing reagents (i.e., nucleases, base editors, prime editors, therapeutic transgenes), or by ex vivo modification of cells and subsequent transplant. While in vivo and ex vivo modification are promising strategies for certain target cell types, it is not an option for many target cells and tissues. Current approaches for delivery to target cells largely rely upon chemical or physical means (e.g., lipofection, electroporation) or the use of engineered polymers or viral vectors, namely recombinant adeno-associated viruses (rAAV) and lentiviruses. These modes of delivery have inherent challenges. For instance, electroporation of primary immune cells typically requires activation for successful delivery of molecules. While activated cells are more amenable to transduction and electroporation, the process activates RNA/DNA cytosolic sensors and leads to RNA/DNA toxicity and low cell viability. Electroporation is only utilized for in vitro or ex vivo studies, and is highly efficient for transformed cancer cells but inefficient for many types of primary cells. The use of viral delivery (in vitro, ex vivo, and in vivo) is hampered by several challenges including cost, timelines for GMP grade virus, immunogenicity, delivery efficiency, and cargo capacity constraints. Importantly, it is difficult to be selective about what target cells receive molecules delivered by viruses or polymers. Accordingly, there remains a need in the art for improved cell-to-cell methods for delivering nucleic acids, gene editing reagents, and therapeutic cargo in vivo.

### SUMMARY OF THE DISCLOSURE

The present invention provides methods for delivering one or more cargo molecules to a target cell via a modified donor cell, compositions, modified donor cells, modified target cells, and methods of use.

In one aspect, the disclosure provides a method for delivering a cargo molecule to a target cell, the method comprising: (a) introducing one or more nucleic acids encoding a fusion protein comprising a transmembrane receptor and the cargo molecule into a donor cell producing a modified donor cell expressing the fusion protein; and (b) co-culturing the modified donor cell with the target cell, whereby a modified target cell comprising the fusion protein of the modified donor cell is obtained. In some embodiments, one or more fusion proteins are introduced into the donor cell.

In yet another aspect, the disclosure provides a modified target cell obtained according to the methods described herein comprising one or more fusion proteins, one or more guide RNAs or combinations thereof.

In one aspect, the disclosure provides a method of treating a subject having a disease, the method comprising administering the modified target cell described herein in an amount effective to treat the disease.

In another aspect, the disclosure provides a genetically modified human donor cell comprising one or more nucleic acids encoding a fusion protein comprising a transmembrane receptor and a cargo molecule as described herein.

In another aspect, the disclosure provides a method of treating a subject with a disease, the method comprising administering the genetically modified human donor cell of described herein in an amount effective to treat the disease.

In another aspect, the disclosure provides a method for delivering one or more cargo molecule to a target cell for gene editing the target cell. The method comprises (a) introducing one or more nucleic acids encoding (i) one or more fusion proteins into a donor cell, wherein each fusion protein comprises a transmembrane receptor and a cargo molecule comprising a gene editing protein, and (ii) one or more guide RNA or DNA encoding a guide RNA, producing a modified donor cell expressing the one or more fusion protein and one or more guide RNAs; and (b) co-culturing the modified donor cell with the target cell, whereby a modified target cell comprising the fusion protein and guide RNAs of the modified donor cell is obtained and the gene editing protein is capable of editing the target cell.

In another aspect, provided herein is a method for delivering a biological molecule (biomolecule) to a target cell. The method can comprise or consist essentially of (a) introducing into a donor cell one or more nucleic acids encoding a fusion protein comprising a transmembrane receptor fused to a cargo biomolecule by a linker comprising one or more furin cleavage sites, wherein the donor cell lacks expression of furin, whereby a modified donor cell comprising the fusion protein is obtained; and (b) co-culturing the modified donor cell with the target cell under conditions that promote trogocytosis between the modified donor cell and the target cell, whereby a modified target cell comprising membrane material and the fusion protein of the modified donor cell is obtained, wherein furin expressed in the modified target cell cleaves the fusion construct at the one or more furin cleavage sites and releases the cargo biomolecule in the modified target cell. The donor cell can be a human monocyte, macrophage, natural killer (NK) cell, cytotoxic T cell, regulatory T cell, B cell, or gamma-delta T cell.

The target cell can be a human cell. The human cell can be a T cell, B cell, CD34+ hematopoietic stem cell (HSC), natural killer cell, a tumor cell, hepatocyte, liver stellate cell, neuron, microglia, fibroblast, keratinocyte, epithelial cell, hair follicle stem cell, or muscle cell, or a progenitor thereof. The fusion protein can be introduced to donor cell's genome at the furin locus, thereby reducing or preventing expression of endogenous furin in the donor cell. The cargo biomolecule can be a protein. The cargo molecule can be a RNA binding protein bound to a cargo RNA molecule of interest. The RNA binding protein can be a MS2 RNA binding protein. The cargo molecule can be a DNA binding protein bound to a cargo DNA molecule of interest. The DNA binding protein can be a Transcription activator-like effector (TALE) or a HUH endonuclease, or a portion thereof. The protein can be a gene editing reagent. The gene editing reagent can be a Cas nuclease, zinc finger nuclease (ZFN), TALEN, base editor, prime editor, transposon/transposase, or integrase. The method can be performed in vivo, in vitro, or ex vivo.

In another aspect, provided herein is a modified target cell obtained according to a method of this disclosure.

In a further aspect, provided herein is a genetically modified human donor cell comprising one or more nucleic acids encoding a fusion protein comprising a transmembrane receptor fused to a cargo biomolecule by a linker comprising one or more furin cleavage sites, wherein the human donor cell is genetically modified such that is it deficient in expression of furin. The human donor cell can be a human monocyte, macrophage, natural killer (NK) cell, cytotoxic T cell, regulatory T cell, B cell, or gamma-delta T cell or other human donor cells that are capable of trogocytosis. In one example, the donor cell is an NK cell. In another example, the donor cell is a T cell. In a further example, the donor cell is a B cell.

The fusion protein can be introduced to the donor cell genome at the furin locus, whereby the modified donor cell does not express endogenous furin. The human donor cell can be further genetically modified to express a chimeric antigen receptor (CAR) comprising a single-chain variant fragment (scFv) specific for a target cell, whereby said genetically-modified human cell expresses said CAR. The ligand-binding domain of the CAR can be specific for CD34 and wherein the target cell is a CD34⁺ hematopoietic stem cell (HSC).

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention and to cover all modifications, equivalents and alternatives. Reference should therefore be made to the claims recited herein for interpreting the scope of the invention.

### BRIEF DESCRIPTION OF THE DRAWINGS

FIG. 1 illustrates an embodiment of molecule delivery from donor cells that lack expression of furin to acceptor cells using furin cleavage sites.

FIG. 2 illustrates trogocytosis delivery of transmembrane proteins.

FIG. 3 illustrates various “cargo” molecules that can be delivered by trogocytosis delivery methods of this disclosure.

FIG. 4 illustrates one embodiment of trogocytosis delivery of transmembrane fusion proteins. In this embodiment, “donor” cells are furin-KO K562 Feeders, “acceptor” cells are NK cells, the transmembrane protein of the fusion protein is CCR7 or Transferrin, and the protein “cargo” of the fusion protein is enhanced GFP (EGFP).

FIG. 5 present flow cytometry data from (in top panels) feeder GFP and CCR7 gating determined using fully-stained non-electroporated Clone9.mbIL21 (do not express GFP/CCR7) and (in lower panels) NK GFP and CCR7 gating determined using fully-stained, non-co-cultured WT NK cells.

FIG. 6 presents flow cytometry data following single cell, viability gating based on CellTrace Violet and CD56 staining.

FIG. 7 demonstrates GFP and CCR7 expression in NK cells following co-culture for various lengths of time.

FIG. 8 demonstrates GFP and CCR7 expression in NK cells following co-culture with donor (feeder) cells and gating by flow cytometry.

FIGS. 9A-9B show exemplary embodiments of TIMIT using (A) K562 feeder cells and NK acceptor cells, and (B) 293T cells and B cells.

FIG. 10 is a western blot depicting the knocking out of furin in the donor cell line.

FIG. 11 is representative flow cytometry analysis demonstrating the expression of the cargo molecule (represented by GFP expression) in the donor cells either normal or furin-KO.

FIG. 12 is representative flow cytometry demonstrating CCR7 expression in the furin containing and furin KO cells containing the CCR7-GFP or TfnR-GFP constructs FIG. 13 are graphs demonstrating the transfer of cargo molecules (i.e., GFP) via the different constructs as percentage GFP and as mean fluorescent intensity (MFI).

FIG. 14 demonstrates the GFP expression in NK cells cultured with either the Furin+vs Furin-KO feeder cells as both percent GFP positive and as mean fluorescent intensity (MFI).

FIG. 15 are graphs demonstrating CCR7 expression in NK cells incubated with the feeder cells (furin+ and furin-KO).

FIG. 16. are graphs demonstrating CCR7 expression in NK cells incubated with the feeder cells (furin+ and furin-KO).

FIG. 17 is a representative flow plot demonstrating the transfer of the fusion protein comprising MS2 binding protein into NK cells.

FIG. 18 shows the expected outcomes for the different feeder cell types.

FIG. 19 depicts the method used to demonstrate the specificity of the methods described herein using a GFP-mRNA handoff experiment.

FIG. 20 is a graph demonstrating that GFP positive NK cells post sort show an increase in number of GFP-expressing NK cells, suggesting increases in GFP which occurs through mRNA translation.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

All publications, including but not limited to patents and patent applications, cited in this specification are herein incorporated by reference as though set forth in their entirety in the present application.

The methods and compositions described herein are based at least in part on the inventors' determination that trogocytosis can be leveraged to transfer intracellular molecules from genetically engineered “donor” cells into primary cell types and pluripotent stem cells. Trogocytosis is the process whereby a “donor” lymphocyte or other cell conjugates to an “acceptor” or “target” cell and transfers small portions of membrane, including cell surface receptors from the donor to target cell. A hallmark of trogocytosis is that transferred membrane proteins retain their orientation and their function until they are recycled via normal membrane turnover.

As described herein, Transfer of Intracellular Molecules via Intercellular Trogocytosis (TIMIT) permits targeted in vitro, ex vivo, and in vivo delivery of molecular cargo (e.g., nucleic acids, proteins, therapeutic agents, small molecules) into target cells, including target primary cells which are usually intractable to conventional delivery methods. Conventional approaches for in vitro and in vivo delivery largely rely upon the use of engineered polymers or viral vectors, namely recombinant adeno-associated viruses (rAAV) and lentiviruses, but the success of these approaches is limited by immunogenicity, vector carrying capacity, and delivery efficiency.

Importantly, it is difficult to be selective about what target cells receive molecules delivered by viruses or polymers, and efficiency, particularly for primary cells, can be very low. The inventors have improved upon existing methods by harnessing the process of trogocytosis to deliver molecules that are fused or tethered to cell surface receptors. In particular, the methods employ donor cells that have been genetically engineered to express a fusion protein comprising a transmembrane receptor and a cargo molecule whereby the cargo molecule is targeted to the donor cell membrane for transfer via trogocytosis. Trogocytosis delivers the cargo-fusion construct with small portions of membrane into an acceptor target cell. In some embodiments wherein the cargo molecule is a protein, the methods employ donor cells that have been genetically engineered to eliminate expression of certain endogenous cleavage molecules such as proteolytic cleavage enzyme, peptidase or furin, whereby the cargo molecules can be fused to a transmembrane receptor and, thus, targeted to the donor cell membrane. Trogocytosis delivers the cargo-fusion construct with small portions of membrane into an acceptor target cell, whereby expression of the appropriate cleavage molecule promotes cleavage and release of the cargo into the acceptor cell.

Advantages of these methods and compositions provided herein are multifold. In particular, the advanced technology of this disclosure can be used for targeted in vitro, in vivo, or ex vivo delivery of various types of cargo to particular target cells. In some embodiments, the cargo comprises one or more gene editing reagents such as Cas nucleases, base editors, and guide nucleic acids. In addition, the methods are particularly well-suited for clinical applications such as targeted for in vivo or ex vivo delivery of therapeutic payloads to tumor cells, pro-survival factors for treatment of degenerative diseases, or enzymes that are deficient as a result of a metabolic disease.

Accordingly, in a first aspect, provided herein are genetically modified donor cells that have been engineered to express a fusion protein via one or more constructs, preferably one nucleic acid construct. The fusion protein comprises a cargo molecule and a transmembrane receptor protein or a portion thereof, wherein the cargo molecule is linked to the transmembrane receptor protein or portion thereof such that when the fusion protein is expressed in the modified donor cell, the cargo-transmembrane receptor fusion proteins will be targeted to the modified donor cell's membrane. In some aspects, the transmembrane receptor protein or portion thereof is fused to the cargo molecule via being expressed by the same nucleic acid sequence. In some embodiments, the genetically modified donor cells are engineered to express two or more fusion proteins to be able to transport two or more cargo molecules to a target cell (e.g., a gene editing agent and one or more guide RNAs, etc.).

When the engineered donor cell is cultured in the presence of an acceptor or target cell that expresses a molecule that binds or brings the donor cell in close proximity to the target cell, trogocytosis can occur between the two cells, in which case the acceptor (target) cell takes up a portion of the donor cell's membrane, including the membrane-associated fusion protein. As used herein, the term “donor cell” refers to the cell that will donate material (e.g., membrane material and membrane-associated material and proteins) by trogocytosis to an acceptor cells when the donor and acceptor cells are co-cultured.

As used herein, the term “acceptor cell” or “target cell” refers to the cells that will receive material (e.g., membrane material and membrane-associated material and proteins) by trogocytosis from the engineered donor cell. By varying the cargo molecule and acceptor (target) cells, genetically modified donor cells of this disclosure are useful for in vivo, in vitro, or ex vivo delivery of molecules of interest to cells, including cells that are difficult to modify or target using conventional methods. Accordingly, genetically modified donor cells of this disclosure are useful for, by way of example, in vivo, in vitro, or ex vivo delivery of gene editing reagents to cells for gene therapy (i.e., genetic correction), genetic manipulation, therapeutic payloads to tumor cells, pro-survival factors for treatment of degenerative diseases, delivery of genetic modifiers that remove or render viruses inactive, or deficient enzymes for treatment of metabolic disease.

In some embodiments, the genetically modified donor cell is used in targeted delivery methods. Any appropriate cell can be used as a donor cell. Appropriate cells include those cells that are amenable to introduction of the fusion construct. Exemplary donor cell types include, without limitation, leukocytes (e.g., monocytes, lymphocytes, granulocytes, and macrophages), transformed cell lines, immortalized cell lines, primary cells, and human pluripotent stem cells, including induced pluripotent stem cells. Exemplary lymphocytes include natural killer (NK) cells, CD4+ and CD8+ T cells, regulatory T cells, and gamma-delta T cells.

The fusion protein expressed in the donor cells by one or more nucleic acid constructs, preferably a single construct that expresses the entire fusion protein. The donor cell is preferably a human donor cell.

The fusion protein comprises a transmembrane receptor or portion thereof and a cargo molecule. The fusion protein can further comprise a linker linking the transmembrane receptor or portion thereof and the cargo molecule. In another embodiment, the fusion protein further comprises one or more furin cleavage sites, and the donor cell expressing the fusion protein will be furin deficient. This allows the fusion protein to be able to be expressed in the donor cell but cleaved once delivered to a target cell by the endogenous furin in the target cell.

The cargo molecule may be any protein to be delivered to the target cell. For delivery of other types of protein cargo to an acceptor cell, the desired protein cargo is expressed in the donor cell as part of the fusion protein. Essentially any protein that can be expressed as a recombinant protein without loss of function can be used for the compositions and methods of this disclosure. It will be understood by ordinarily skilled practitioners in the field that protein cargo may be selected based on the desired effect or response to be elicited in the acceptor cell, which will receive the cargo via trogocytosis. Suitable cargo molecules may be gene editing proteins, therapeutic proteins, cytokines, antibodies, protein fragments, RNA binding proteins, DNA binding proteins, etc. Any suitable cargo molecule can be used in the practice of the present invention and is not limited to those described as examples herein. For example, in one embodiment, the fusion protein comprises a cargo molecule able to alters cytokine or chemokine production (e.g., production of IFNγ, TNFα, IL-17, IL-22, MIP-1α (CCL3), MIP-1β (CCL4), and/or RANTES (CCLS)), alters a cytokine receptor expression (e.g., but not limited to, IL-2R, IL-12R, IL-18R, IL-21R, etc.) or a chemokine receptor (e.g., but not limited to, CCR1, CCR2, CCR3, CCR4, CCR5, CCR6, CCR7, CXCR1, CXCR2, CXCR3, CXCR4, CXCR5, CXCR6, etc.).

Other cargo molecules include hormone receptors, signaling ligands, CAR/ScFV can also be used and this list is not to be considered exhaustive of the potential cargo proteins used in the practice of the present invention.

In one embodiment, the protein may be an RNA or DNA binding protein allowing for the ability to transport an RNA or DNA molecule of interest to the target cell. In the instance in which the cargo molecule is a RNA or DNA binding protein, the fusion protein may contain but does not require to contain a protease cleavage (e.g., furin cleavage) site as the RNA or DNA being transported by the RNA or DNA binding protein of the fusion protein does not need to be cleaved once delivered to the target cell for activity.

Thus, in some embodiments, the cargo molecule will be based on the type of cargo cell being targeted and/or the desired effect to be elicited in the target cell. For instance, if the acceptor cell is a natural killer (NK) cell for which it would be advantageous to increase expression of CCR7, the protein cargo may be a chemokine receptor such as CCR7. Uptake of the CCR7 “cargo” via trogocytosis using the genetically modified cells of this disclosure would achieve CCR7 expression in the acceptor NK cells which, without being bound by any particular theory or mode of action, would increase targeting of the cells toward lymph nodes for adoptive immunotherapy for various cancers.

In some embodiments, the transmembrane receptor and cargo portions are fused together with a linker. In one embodiment, the linker is a flexible linker. In another embodiment, the linker preferably comprises one or more furin cleavage sites. As used herein, a “peptide linker” or “linker” is a polypeptide typically ranging from about 2 to about 150 amino acids in length, which is designed to facilitate the functional connection of two polypeptides into a linked fusion polypeptide. The term functional connection denotes a connection that facilitates proper folding of the polypeptides into a three-dimensional structure that allows the linked fusion polypeptide to mimic some or all of the functional aspects or biological activities of the proteins from which its polypeptide constituents are derived. The term functional connection also denotes a connection that confers a degree of stability required for the resulting linked fusion polypeptide to function as desired. In each particular case, the preferred linker length will depend upon the nature of the polypeptides to be linked and the desired activity of the linked fusion polypeptide resulting from the linkage. Generally, the linker should be long enough to allow the resulting linked fusion polypeptide to properly fold into a conformation providing the desired biological activity. Most suitably, linkers comprises flexible amino acids (glycines and serines) that allow for flexibility between the transmembrane receptor and cargo proteins. Suitable linkers are known in the art, for example, (GSGS)n, (GGSS)n, (GS)n, (G)n, (S)n, etc. Further, the linker may include one or more protease cleavage sites as described herein, for example, at least one or more furin cleavage sites.

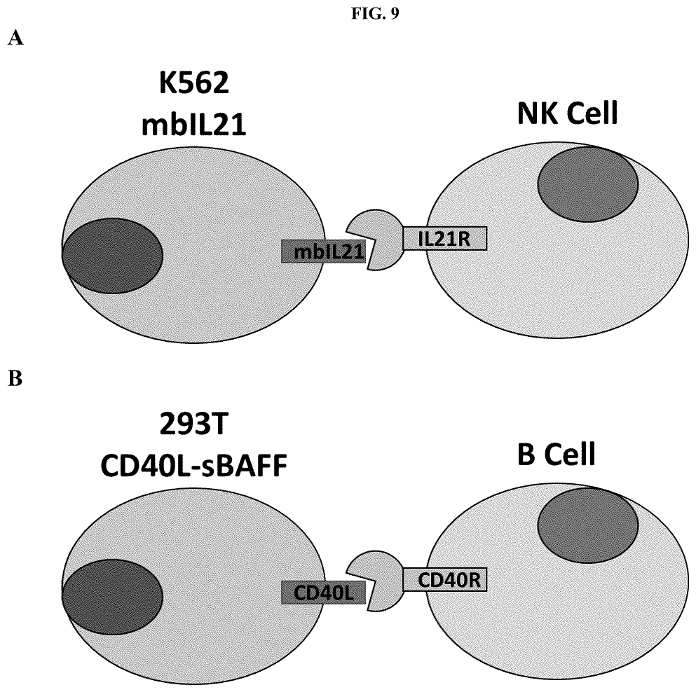

In some embodiments, genetically modified donor cells of this disclosure are further modified to express a chimeric antigen receptor (CAR) that is specific to a cell surface marker on a target (acceptor) cell of interest. As used herein, the term “chimeric antigen receptor (CAR)” (also known in the art as chimeric receptors and chimeric immune receptors) refers to an artificially constructed hybrid protein or polypeptide comprising an extracellular antigen binding domains of an antibody (e.g., single chain variable fragment (scFv)) operably linked to a transmembrane domain and at least one intracellular domain. Generally, the antigen binding domain of a CAR has specificity for a particular antigen expressed on the surface of a target cell of interest. For example, a cell can be engineered to express a CAR specific for molecule expressed on the surface of a particular cell (e.g., a tumor cell, B-cell lymphoma). In some cases, a natural killer (NK) cell or cytotoxic T cell can be modified to express a CD34-specific CAR. In such cases, when the modified NK cell is co-cultured with target cells expressing CD34, the target cells will activate the NK cell via the CD34-CAR. Other surface markers of HSCs to specifically target HSCs with CAR-expressing modified lymphocytes include, without limitation, cKIT, CD90, and CD49f. Referring to FIG. 9A, in some embodiments, TIMIT is performed using K562 feeder cells and NK cells. In such embodiments, the K562 feeder cells may express mbIL21 for NK cell expansion, and the mbIL21:IL21R interaction should induce trogocytosis. Referring to FIG. 9B, in some embodiments, TIMIT is performed using 293T cells and B cells. In such embodiments, the 293T feeder cells may express CD40L/sBAFF for B cell expansion, and the CD40L:CD40R interaction should induce trogocytosis.

In some cases, genetically engineered donor cells of this disclosure comprise at least two transgenes (encoding the CAR and the fusion construct). In some cases, the donor cell is further genetically engineered to knockout a particular gene. As described herein, donor cells are preferably furin-deficient so that the fusion construct is not cleaved in the donor cell prior to co-culture with acceptor cells. In some cases, such engineered cells are obtained by gene editing of primary human cells by any appropriate means. For example, multiplexed editing can be performed in primary human monocytes, lymphocytes, NK cells, or other cells. In other cases, the engineered donors are obtained by performing genetic manipulation in pluripotent stem cells and then differentiating the genetically modified pluripotent cells in vitro to obtain the differentiated cell type of interest (e.g., an engineered leukocyte). Exemplary protocols for directing differentiation of pluripotent stem cells to various cell types including leukocytes are known in the art. For instance, protocols for directing differentiation of pluripotent stem cells to NK cells are described, for example, in Li et al. (Cell Stem Cell 23, 181-192.e5 (2018)) and Shankar et al. (Stem Cell Res Ther 11, (2020)), each of which is incorporated herein by reference.

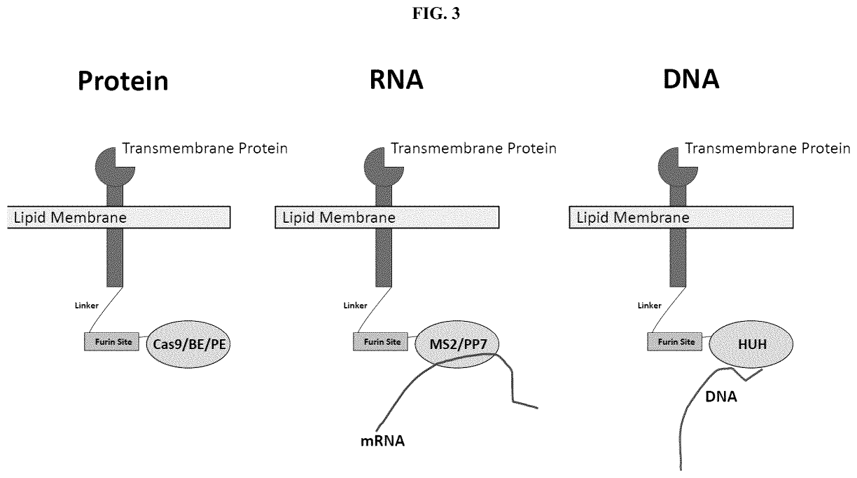

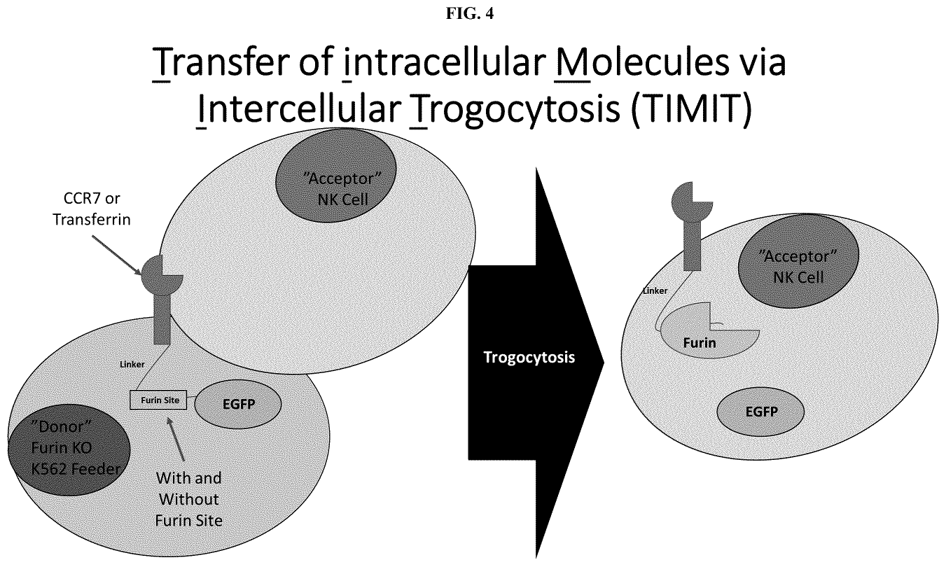

For delivery of RNA molecules using the trogocytosis-based in vivo or in vitro delivery methods of this disclosure, a RNA binding protein is fused to a transmembrane receptor protein or a membrane-binding portion thereof. In such embodiments, the RNAs to be transferred may be engineered to comprise one or more sequences that are binding regions for the specific RNA binding protein. In some embodiments, the RNA binding protein is MS2 as illustrated in FIG. 3. In such embodiments, RNAs to be transferred may be configured to contain one or more MS2 RNA loops (e.g., acatgaggatcacccatgt SEQ ID NO:15) for recognition of and binding to MS2 (Bardwell and Wickens, “Purification of RNA and RNA-protein complexes by an R17 coat protein affinity method”, Nucleic Acids Res. 1990 Nov. 25; 18(22): 6587-6594, PMID 1701242, which is incorporated herein by reference). By way of example, the MS2-transmembrane receptor fusion protein is useful to transfer Cas9 mRNA and gRNA that comprise one or more MS2 binding sequences. Other exemplary RNA binding proteins include, without limitation, the RNA-binding coat proteins of phages PP7 (Lim et al. “Translational repression and specific RNA binding by the coat protein of the Pseudomonas phage PP7”, J Biol Chem. 2001 Jun. 22; 276(25):22507-13, PMID 11306589, GA (Gott et al. “RNA binding properties of the coat protein from bacteriophage GA”, Nucleic Acids Research, Volume 19, Issue 23, 11 Dec. 1991, Pages 6499-6503, PMID 1754387), and QP (Lim et al. “The RNA-binding site of bacteriophage Qbeta coat protein”, J Biol Chem. 1996 Dec. 13; 271(50):31839-45. PMID 8943226) each of which are incorporated by reference herein.

Thus, the donor cells may further express one or more RNA of interest to be bound to the RNA binding protein and delivered to the target cells. Suitable RNA molecules of interest may be therapeutic RNAs (e.g., siRNA, mRNA). In some embodiments, the RNA is an mRNA encodes for a gene editing protein or a therapeutic protein of interest. In some embodiments, the RNA is a guide RNA for gene editing of the target cell. In some embodiments, the RNA is a guide RNA to disrupt an endogenous gene. In another embodiment, the RNA is a guide RNA to repair an endogenous gene comprising a mutation. In a further embodiment, the RNA is a guide RNA for editing an exogenous DNA within the cell, e.g., a pathogenic gene, for example, a viral gene. In some embodiments, the RNA is a guide RNA for editing viral DNA, thereby disrupting viral gene activity.

For delivery of DNA molecules using the trogocytosis-based delivery methods of this disclosure, a specific DNA binding protein is fused to a transmembrane receptor protein or a membrane-binding portion thereof. DNA binding proteins bind to single- or double-stranded DNA in a sequence-specific (as in the case of transcription factors and DNA nucleases) or sequence non-specific manner (as in the case of polymerases and histones). In some embodiments, the DNA binding protein is a Transcription activator-like effector (TALE). For example, the use of TALE is described in ACS Synth. Biol. 2014, 3, 10, 708-716, Feb. 5, 2014, //doi.org/10.1021/sb400137b, incorporated by reference, which discloses a 32 amino acid repeat (SEQ ID NO: 16-LTPEQVVAIASHDGGKQALETVQRLLRVCQAHG). In other embodiments, the DNA binding protein is a DNA conjugating enzyme such as a member of the HUH endonuclease superfamily. HUH endonucleases contain a conserved pair of metal-coordinating histidines (H) separated by a hydrophobic residue (U), and are known to recognize and bind DNA hairpin structures. HUH-endonuclease domains can be used as fusion tags for covalent linkages between proteins and DNA. For example, purified HUH proteins form stable covalent bonds in vitro with synthetic oligonucleotides bearing a specific sequence at the origin of replication (ori), and do not require incorporation of modified bases into the DNA to be bound. Accordingly, HUH proteins can be used as fusion tags in a fusion protein with a transmembrane receptor (or membrane-associated portion thereof) without disruption of the transmembrane receptor's function. HUH-protein tags suitable for use in fusion constructs of this disclosure are set forth in Lovendahl et al. (J. Am. Chem. Soc. 2017, 139(20):7030-7035, incorporated by reference in its entirety) (MAKSGNYSYKRWVFTINNPTFEDYVHVLEFCTLDNCKFAIVGEEKGANGTPHLQGFLN LRSNARAAALEESLGGRAWLSRARGSDEDNEEYCAKESTYLRVGEPVSKGRSS, SEQ ID NO: 17). As described by Lovendahl et al., HUH-tagged recombinant proteins can be used to covalently tether a single stranded DNA (ssDNA) containing an appropriate HUH-domain target sequence to Cas9. By designing cargo DNA to contain a HUH-domain target sequence, the cargo DNA will bind to the HUH protein tag of the fusion construct. As illustrated in FIG. 3, the fusion construct can comprises a furin-site containing linker such that, following trogocytosis between the donor cell and acceptor cell, the HUH protein and bound cargo DNA are released into the furin-expressing acceptor cell. Types of DNA molecules that can be introduced into acceptor cells according to these methods include, without limitation, single stranded DNA, double stranded DNA, linear DNA, circular DNA, plasmid DNA, and DNA transposons for homologous recombination. In addition, the delivery methods of this disclosure may be used to deliver transposase fusions that can bind transposon inverted terminal repeat sequences (ITRs).

Other exemplary DNA binding proteins that can be used for the genetically modified cells of this disclosure include, without limitation, TALEs, CRISPR enzymes, proteins comprising a zinc finger nuclease (ZFN) binding domain, proteins comprising a transposase binding domain (for transposon plasmids), proteins comprising a Tet operon, and tetracycline transactivator (tTA) proteins. Additional DNA binding moieties that can be used include, without limitation, GAL4 DNA binding domains (GAL4 motif: 5′-CGG-(N11)-CCG-3′), PPR1 binding domains (PPR1 motif: 5′-CGG-(N6)-CCG-3′) and synthetic transcription factor binding domains. In some cases, the DNA binding moiety is monomeric streptavidin for biotinylated DNA templates. Suitable CRISPR enzymes are known and understood in the art to be able to be used in the practice of this invention.

Transmembrane Receptor

Any appropriate cell surface receptor can be used in fusion constructs of this disclosure as the transmembrane receptor or a portion thereof that will tether the cargo molecule to the lipid membrane of the donor cell. As used herein, the terms “cell surface receptor” and “transmembrane receptor” (also known as membrane receptors) are used interchangeably herein and refer to a protein that is attached to and/or embedded in the plasma membrane of a cell. Transmembrane receptors generally span the plasma membrane, with the extracellular domain of the protein having the ability to bind to a ligand and the intracellular domain having an activity (such as a protein kinase) that can be altered (either increased or decreased) upon ligand binding. In some embodiments, the transmembrane receptor is a natural or synthetic membrane bound receptor. Preferably, the fusion construct is designed so the cargo biomolecule is fused to the transmembrane receptor on the intracellular side of the donor cell membrane. Exemplary transmembrane receptors include, without limitation, G protein-coupled receptors, ion channel-linked receptors, enzyme-linked receptors, receptor tyrosine kinases, receptor serine/threonine kinases, and cytokine receptors (e.g., receptors for the interferon family for interleukin, erythropoietin, G-CSF, GM-CSF, tumor necrosis factor (TNF) and leptin receptors).

In some embodiments, comprises a cargo molecule and a transmembrane receptor protein or a portion thereof, wherein the cargo molecule is linked to the transmembrane receptor protein or portion thereof via a linker. In one embodiment, the linker comprises one or more furin cleavage sites between the transmembrane receptor and cargo molecule such that the linker is capable of being cleaved and releasing the cargo molecule from the transmembrane receptor protein in the target cell.

The fusion protein is expressed by one or more nucleic acid constructs in the donor cell. The term “nucleic acid construct,” “construct” and “expression construct” refer to a polynucleotide sequence encoding the protein of interest (e.g., fusion protein) and a promoter operably connected to a polynucleotide. The polynucleotide sequence may comprise heterologous backbone sequence. The nucleic acid sequence may be a vector. As used herein, the term “vector” refers to a nucleic acid molecule capable of propagating another nucleic acid to which it is linked. Suitable vectors for use with the present invention comprise a promoter operably connected to a polynucleotide sequence encoding the fusion peptide described herein. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” (or simply, “vectors”). The term vector encompasses “plasmids”, the most commonly used form of vector. Plasmids are circular double-stranded DNA loops into which additional DNA segments (e.g., those encoding inhibitory Fis1 peptides) may be ligated. However, other forms of expression vectors, such as viral vectors (e.g., replication defective retroviruses, adenoviruses and adena-associated viruses), may also be used with the present invention. Certain vectors are capable of autonomous replication in a host cell into which they are introduced (e.g., bacterial vectors having a bacterial origin of replication and episomal mammalian vectors). Other vectors may be integrated into the genome of a host cell upon introduction into the host cell, and are thereby replicated along with the host genome. In one embodiment, the vectors comprise viral vectors that use viral machinery to carry the peptide to be expressed in a host cell. The vectors may also comprise appropriate control sequences that allow for translational regulation in a host cell. In some embodiments, the vectors further comprise the nucleic acid sequences for one or more additional proteins. In some embodiments, the vectors further comprise additional regulatory sequences, such as signal sequences.

In some embodiments, the vectors of the present invention further comprise heterologous backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of the encoded peptide. Many commonly used expression vectors and plasmids contain non-human nucleic acid sequences, including, for example, CMV promoters.

Any appropriate gene modification techniques can be used to obtain genetically modified cells comprising the targeted disruptions described herein. In some embodiments, the cargo molecule, including, for example, gene editing components including a base editor and a guide molecule can be delivered to a cell (e.g., a pluripotent stem cell) in vitro, ex vivo, or in vivo. In some cases, a viral or plasmid vector system is employed for delivery of base editing components described herein. Preferably, the vector is a viral vector, such as a lenti- or baculo- or preferably adeno-viral/adeno-associated viral (AAV) vectors, but other means of delivery are known (such as yeast systems, microvesicles, gene guns/means of attaching vectors to gold nanoparticles) and are contemplated. In certain embodiments, nucleic acids encoding gRNAs and base editor fusion proteins are packaged for delivery to a cell in one or more viral delivery vectors. Suitable viral delivery vectors include, without limitation, adeno-viral/adeno-associated viral (AAV) vectors, lentiviral vectors. In some cases, non-viral transfer methods as are known in the art can be used to introduce nucleic acids or proteins in mammalian cells. Nucleic acids and proteins can be delivered with a pharmaceutically acceptable vehicle, or for example, encapsulated in a liposome. Other means of delivery are known (such as yeast systems, microvesicles, gene guns/means of attaching vectors to gold nanoparticles) and are contemplated. In some cases, cells are electroporated for uptake of gRNA and base editor (e.g., BE3, BE4, ABE). In some cases, DNA donor template is delivered as Adeno-Associated Virus Type 6 (AAV6) vector by addition of viral supernatant to culture medium after introduction of the gRNA, base editor, and vector by electroporation.

In some embodiments, components of a fusion construct described herein can be delivered to a cell in vitro, ex vivo, or in vivo.

In some embodiments, a viral or plasmid vector system is employed for delivery of nucleic acids encoding a fusion protein as described herein. Preferably, the vector is a viral vector, such as a lenti- or baculo- or preferably adeno-viral/adeno-associated viral (AAV) vectors, but other means of delivery are known (such as yeast systems, microvesicles, gene guns/means of attaching vectors to gold nanoparticles) and are contemplated. In certain embodiments, nucleic acids encoding fusion proteins are packaged for delivery to a cell in one or more viral delivery vectors. Suitable viral delivery vectors include, without limitation, adeno-viral/adeno-associated viral (AAV) vectors, lentiviral vectors. In some cases, non-viral transfer methods as are known in the art can be used to introduce nucleic acids or proteins in mammalian cells. Nucleic acids and proteins can be delivered with a pharmaceutically acceptable vehicle, or for example, encapsulated in a liposome. Other means of delivery are known (such as yeast systems, microvesicles, gene guns/means of attaching vectors to gold nanoparticles) and are contemplated. In some cases, cells are electroporated for uptake of nucleic acids encoding fusion proteins. In some cases, DNA donor template is delivered as Adeno-Associated Virus Type 6 (AAV6) vector by addition of viral supernatant to culture medium after introduction of the gRNA, Cas enzyme or base editor, and vector by electroporation.

In some embodiments, it is advantageous to use chemically modified nucleic acids having increased stability. For example, nucleic acids can be chemically modified to comprise 2′-O-methyl phosphorothioate modifications on at least one 5′ nucleotide and at least one 3′ nucleotide of each gRNA. In some cases, the three terminal 5′ nucleotides and three terminal 3′ nucleotides are chemically modified to comprise 2′-O-methyl phosphorothioate modifications.

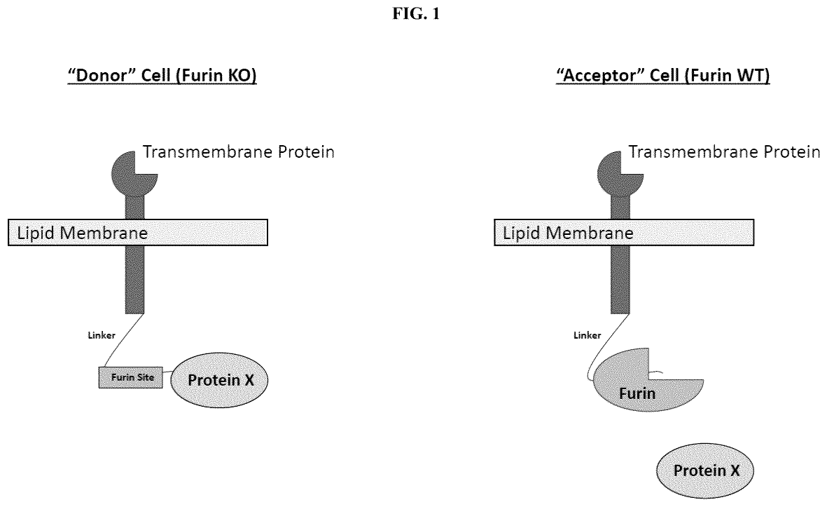

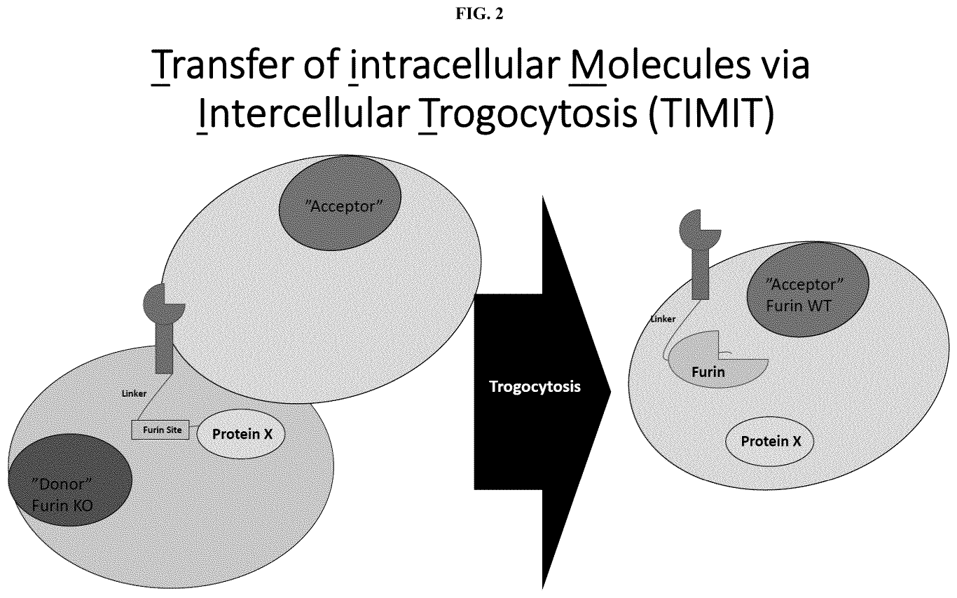

In one embodiment, the donor cell lacks expression of a proteinase, for example, furin (e.g., is a furin-knockout cell) and the donor cell expresses a fusion protein comprising the transmembrane receptor and cargo molecule separated by a linker comprising at least one protease cleavage site (e.g., furin cleavage site). Furin is a calcium-dependent serine endoprotease that can efficiently cleave precursor proteins at their paired basic amino acid processing sites to yield functional protein. In the absence of functional furin, proteins containing furin consensus cleavage sites are not cleaved. When the donor cell is genetically modified to lack furin, it will be advantageous to use a linker that comprises a furin cleavage sequence (“furin cleavage motif”) to join the cargo and transmembrane receptor in the fusion protein. See FIG. 1. For genetically modified donor cells of this disclosure, upon introduction of the expression construct, a furin-deficient donor cell will express the fusion construct and the resulting fusion protein is localized to the intracellular side of the donor cell's membrane. Accordingly, appropriate cells for donor cells are amenable to genetic modification to eliminate endogenous furin expression. As illustrated in FIGS. 1 and 2, upon delivery of the membrane-localized fusion protein to an acceptor target cell via trogocytosis, endogenous expression of furin in the acceptor target cell will lead to cleavage of the fusion protein at the furin cleavage site and release of the cargo portion of the fusion protein from the transmembrane receptor portion.

While furin-deficient cells and furin cleavage sites are exemplified herein, it will be understood by ordinarily skilled practitioners in the field that other proteolytic cleavage mechanisms can be used in place of furin. For instance, it may be advantageous in some embodiments to employ the intramembrane protease γ-secretase (gamma secretase), which is a multi-subunit protease complex that cleaves single-pass transmembrane proteins via its catalytic subunit, presenilin. In such cases, the donor cell may be genetically modified to lack expression of γ-secretase (e.g., is a γ-secretase-knockout cell). Other proteolytic cleavage or peptidasemechanisms that can be used in place of furin or gamma secretase include, without limitation, mechanisms that employ human serine proteases, human cysteine proteases, human aspartyl proteases, human metalloproteases, threonine proteases, and proteases of other species (e.g., non-human metalloproteases or non-human cysteine, serine, or aspartyl proteases). Exemplary proteases are listed at sinobiological.com/category/human-serine-proteases on the World Wide Web.

For delivery of proteins that are gene editing reagents, a Cas enzyme or another gene editing enzyme is fused to a transmembrane receptor protein or a membrane-binding portion thereof. In another embodiment, the mRNA encoding the gene editing reagents may be the mRNA of interest, e.g., the gene editing reagents 9Cas enzyme, guide RNA, etc.) that bind to a fusion protein comprising an RNA binding sequence, as described herein. Any Cas enzyme can be used according to the methods of this disclosure. The terms “Cas” and “CRISPR-associated Cas” are used interchangeably herein. The Cas enzyme can be any naturally-occurring nuclease as well as any chimeras, mutants, homologs, or orthologs. In some embodiments, one or more elements of a CRISPR system is derived from a particular organism comprising an endogenous CRISPR system, such as Streptococcus pyogenes (SP) CRISPR systems or Staphylococcus aureus (SA) CRISPR systems. The CRISPR system is a type II CRISPR system and the Cas enzyme is Cas9 or a catalytically inactive Cas9 (dCas9). Other non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. A comprehensive review of the Cas protein family is presented in Haft et al. (2005) Computational Biology, PLoS Comput. Biol. 1:e60, incorporated by reference in its entirety with regard to the Cas proteins able to be used in the practice of the present invention. At least 41 CRISPR-associated (Cas) gene families have been described to date, and it is contemplated they could be used in the methods described herein.

As used herein, “base editors” are fusion proteins that comprise a Cas nickase domain or catalytically dead Cas protein fused to a deaminase. As in CRISPR-based gene editing, base editors are targeted to specific gene sequences using a guide nucleic acid. However, unlike CRISPR, base editing does not generate double-stranded DNA breaks, making it a safer alternative to Cas nuclease-based methods. Instead, base editing uses the deaminase enzyme to modify a single base without altering the bases around it. There are two classes of base editors: cytosine base editors (CBEs) and adenine base editors (ABEs). CBEs comprise a cytidine deaminase that converts cytidine to uridine within a small editing window near the protospacer adjacent motif (PAM) site. Uridine is subsequently converted to thymidine through base excision repair, creating a cytosine (C) to thymine (T) change (i.e., a guanosine to adenine change on the opposite strand). ABEs comprise an adenine deaminase, which creates an adenine (A) to guanosine (G) change. When a CBE is utilized, to prevent cells from repairing the modified base and encourage the cell to use the edited strand as a template for mismatch repair, a uracil DNA glycosylase inhibitor (UGI) is used to block base excision repair. In some embodiments, a UGI domain is included as part of the base editor fusion protein. In other embodiments, the UGI domain is provided to the cell as a separate component. Researchers have developed third and fourth generation base editors with improved efficiency. For example, the third generation CBE base editor BE3 (i.e., base editor 3) uses a Cas9 nickase to nick the unmodified DNA strand so that it appears “newly synthesized” to the cell, forcing the cell to repair the DNA using the deaminated strand as a template, whereas fourth generation base editors systems (i.e., base editor 4 (BE4)) employ two copies of base excision repair inhibitor UGI. In some embodiments, a BE3 or BE4 cytosine base editor is used in the methods of the present invention. In other embodiments, a CBE comprising a different deaminase, such as hA3A-BE4, hA3G-BE4, evoFERNY-BE4, or evoCDA-BE4, is used. In other embodiments, an ABE base editor, such as ABE6.3, ABE7.10, ABE8e, or ABE8.20 is used. In some embodiments, the base editor enzymes are mutated or modified to confer a desired functionality such as reduced guide-independent off-target editing, reduced guide-dependent off-target editing, an altered editing window, an altered editing context preference, an altered target site specificity, or more precise target editing. Other gene editing components such as base editors and other nucleases can be used in fusion constructs to be introduced into donor cells. For example, in some embodiments, the protein construct can comprise a Cas nuclease, hADARd^{E>Q}, APOBEC cytidine deaminase, MutY DNA glycosylase, or apurinic endonuclease, or a homolog or ortholog of a particular enzyme. While these enzymes are exemplary of suitable base editors and nucleases for use in the disclosed systems and methods a skilled artisan will recognize a range of base editors and nucleases are suitable for use, and a skilled artisan will know how to appropriately select a suitable base editor or nuclease. In other embodiments, gene editing cargo can be, for example, a targeted nuclease (e.g., ZFN, TALEN, CRISPR), a prime editor, a transposon/transposase, or an integrase.

For example, in some embodiments, the cargo molecule is a Cas9 protein, an RNA encoding Cas9, a guide nucleic acid (gNA) or guide RNA or combinations thereof. In one embodiment, the donor cell expresses both a fusion protein comprising the gene editing protein, such as, Cas9 protein, and a fusion protein comprising a RNA binding protein sequence, and also expresses one or more guide RNA that can bind to the RNA binding protein sequence. Thus, both the gene editing protein or Cas9 and one or more guide RNA can be expressed and transferred from the donor cell to the target cell to act on the target cell (e.g., gene edit the target cell).

A “guide RNA (gRNA)” is an RNA molecule that targets an enzyme to a specific genomic sequence via complementary base pairing. The gRNAs used with the present invention comprise a sequence that is complementary to a target sequence within the genome of the target cell. The complementary portion of a gRNA comprises at least 10 contiguous nucleotides, and often comprises 17-23 contiguous nucleotides that are complementary to the target sequence. The complementary portion of the gRNA may be partially or wholly complementary to the target sequence. In some embodiments, the gRNA is from 20 to 120 bases in length, or more. In certain embodiments, the gRNA can be from 20 to 60 bases, 20 to 50 bases, 30 to 50 bases, or 39 to 46 bases in length. Various online tools and software environments can be used to design an appropriate gRNA for a particular application. In some embodiments, the gRNA is a chemically modified gRNA. For example, the gRNA may be chemically modified to decrease a cell's ability to degrade the gRNA. Suitable chemically modified gRNAs may include one or more of the following modifications: 2′-fluoro (2′-F), 2′-O-methyl (2′-O-Me), S-constrained ethyl (cEt), 2′-O-methyl (M), 2′-O-methyl-3′-phosphorothioate (MS), and/or 2′-O-methyl-3′-thiophosphonoacetate (MSP). In some embodiments, the gRNA is composed of two molecules that base pair to form a functional gRNA: one comprising the region that binds to the base editor and one comprising a targeting sequence that binds to the target site. Alternatively, the gRNA may be a single molecule comprising both of these components, i.e., a single guide RNA (sgRNA).

As result of the genetic knockout, the genetically modified donor cell exhibits reduced expression of furin relative to unmodified control cells. As used herein, the term “reduced expression” refers to any reduction in the expression of an endogenous polypeptide in a genetically modified cell when compared to a control cell. The term reduced can also refer to a reduction in the percentage of cells in a population of cells that express an endogenous polypeptide (i.e., an endogenous furin polypeptide) when compared to a population of control cells. Such a reduction may be up to 5%, 10%, 20%, 30%, 40%, 50%, 60%, 70%, 80%, 90%, 95%, or up to 100%.

Accordingly, the term “reduced” encompasses both a partial knockdown and a complete knockdown of the endogenous polypeptide. In preferred embodiments, the genetically modified donor cells exhibits a complete or near complete knockdown of furin expression.

As used herein, the terms “genetically modified” and “genetically engineered” are used interchangeably and refer to a prokaryotic or eukaryotic cell that includes an exogenous polynucleotide, regardless of the method used for insertion. In some cases, the cell has been modified to comprise a non-naturally occurring nucleic acid molecule that has been created or modified by the hand of man (e.g., using recombinant DNA technology) or is derived from such a molecule (e.g., by transcription, translation, etc.). A cell that contains an exogenous, recombinant, synthetic, and/or otherwise modified polynucleotide is considered an engineered cell.

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration that resulted in the genetically-modified cell; (b) a cell of the same genotype as the genetically-modified cell but that has been transformed with a null construct (i.e., with a construct that has no known effect on the trait of interest); or, (c) a cell genetically identical to the genetically-modified cell but that is not exposed to conditions or stimuli or further genetic modifications that would induce expression of altered genotype or phenotype.

### Methods

In another aspect, provided herein are methods for in vivo or in vitro delivery of molecules (e.g., protein, nucleic acids, gene editing reagents, therapeutic payload) to a target cell via donor cells and/or via in vitro-derived target cells. In one embodiment, the methods are used in vitro. In another embodiment, the methods are used in vivo.

Unlike conventional methods which use viruses or engineered polymers to achieve in vivo delivery, the methods of this disclosure use genome-engineered donor cells that can identify specific cell types and deliver molecules to such target cells. It is difficult to be selective about which cells receive molecules when using viruses or polymers in conventional methods, and efficiency can be very low. Accordingly, the methods of this disclosure provide for selective, targeted delivery of molecules in the in vivo setting and, thus, are advantageous over conventional methods.

In one embodiment, the disclosure provides a method for delivering a cargo molecule to a target cell, the method comprising: (a) introducing one or more nucleic acids encoding a fusion protein comprising a transmembrane receptor and the cargo molecule into a donor cell producing a modified donor cell expressing the fusion protein; and (b) co-culturing the modified donor cell with the target cell, whereby a modified target cell comprising the fusion protein of the modified donor cell is obtained. In one embodiment, the donor cell may be contacting the target cell in vitro. In another embodiment, the donor cell may be contacting the target cell in vivo. Suitable methods of in vivo delivery are known in the art, including, for example, systemic administration or local administration known in the art and include but are not limited to, for example, intravenous administration, intra-arterial administration, intratumoral administration, etc.

In some embodiments, the target cell is a cell that expresses a molecule that specifically binds to the transmembrane receptor portion of the fusion protein expressed in the donor cell. In some aspects, as described above, the fusion protein further comprises a linker between the transmembrane receptor and cargo molecule. In some aspects, the linker comprises one or more furin cleavage sites, and the donor cell lacks or has reduced furin expression. In some aspects, the donor cell is a human monocyte, macrophage, natural killer (NK) cell, cytotoxic T cell, regulatory T cell, B cell, or gamma-delta T cell, and the target cells are human cells selected from T cell, B cell, CD34+ hematopoietic stem cell (HSC), natural killer cell, a tumor cell, hepatocyte, liver stellate cell, neuron, microglia, fibroblast, keratinocyte, epithelial cell, hair follicle stem cell, and muscle cell or a progenitor thereof.

In some embodiments, the method comprises introducing a fusion protein comprising an RNA binding protein into the donor cell. In some embodiments, the fusion protein comprises one or more copies of the RNA binding molecule. In some embodiments, the one or more RNA binding molecule comprises the RNA binding portion of the bacteriophage M2 protein. In some embodiments, in addition to the fusion protein, a construct capable of expressing one or more RNA molecules of interest are introduced into the donor cell, in some embodiments, the RNA molecules are guide RNA molecules for gene editing the target cell.

In another embodiment, the method comprises introducing a fusion protein comprising a DNA binding protein and a DNA molecule of interest capable of binding the DNA binding protein into the donor cell. In some aspects, the DNA binding protein is a transcription activator-like effector (TALE) or a HUH endonuclease, or a portion thereof.

In further embodiments, the donor cell lacks expression of furin, and optionally the fusion protein comprises one or more furin cleavage sites between the transmembrane receptor and cargo molecule. This allows for the modified target cell exogenous furin to cleave at the one or more furin cleavage sites, releasing the cargo molecule from the membrane bound transmembrane domain when transferred into the target cell. In some embodiments, the method further comprises prior to step (a), genetically modifying the donor cell to lack expression of furin. Suitable methods of genetically engineering the donor cell to lack furin are described herein, and include, for example, gene editing to knock down or knock out furin. In another embodiment, step (a) comprises the introducing the one or more nucleic acids into the furin locus of the donor cell thereby reducing or preventing expression of endogenous furin in the donor cell and creating the furin⁻ cell.

In some embodiments, the method comprises a fusion protein wherein the cargo molecule is a protein. In some aspects, the protein is a gene editing reagent. In some embodiments, the cargo molecule is a gene editing reagent selected from a Cas nuclease, zinc finger nuclease (ZFN), TALEN, base editor, prime editor, transposon/transposase, or integrase.

As stated, the method of making a modified target cell described herein can be performed in vivo, in vitro, or ex vivo. In some embodiments, step (a) is performed in vitro or ex vivo, and step (b) is performed either in vivo, in vitro, or ex vivo. In some embodiments, both step a and b are preformed ex vivo or in vitro. In another embodiment, step a is performed in vitro or ex vivo In one embodiment, the donor cell is a lymphocyte cell, and the transmembrane receptor is a CCR7 protein or binding fragment thereof. In another embodiment, the cargo molecule is a gene editing agent as described herein.

In another embodiment, modified target molecules obtained according to the methods described herein are produced. The modified target molecules comprise one or more exogenous cargo molecules from the donor cell. In some embodiments, the cargo molecule is a protein. In other embodiments, the cargo molecule is a RNA binding protein bound to an RNA of interest to deliver to the target cell. In a further embodiment, the cargo molecule is a DNA binding protein that is bound to a DNA of interest.

In some embodiments, the in vivo delivery methods achieves delivery of proteins to the target cell. In such cases, the method for in vivo delivery of a protein to a target cell comprises introducing an expression construct into a donor cell, where the expression construct comprises nucleic acid sequence encoding a transmembrane receptor protein fused to a cargo molecule (e.g., protein of interest). Preferably in some embodiments, the donor cell lacks expression of furin (e.g., is a furin-knockout cell). Upon introduction of the expression construct, the donor cell expresses the transmembrane protein-target fusion protein on the intracellular side of the donor cell's membrane. Donor cells will be engineered for specific interaction of immune cell types based on ligand and receptor molecules. In some embodiments, the donor cell is a genetically modified K562 cell, a transformed B cell (raji), a transformed monocyte (THP1), or transformed T cell (Jurkat), or other transformed cell. In one example, the method comprises introducing an expression construct into a furin-knockout donor cell. In other examples, primary human cells are donor cells. In another embodiment, an established cell line is a donor cell. I a further embodiment, a donor cell is derived from pluripotent stem cells. In one embodiment, the donor cell is a B cell. In another embodiment, the donor cell is a T cell or NK cell.

The method further comprises co-culturing the genetically modified donor cells that express the fusion construct with acceptor target cells. Engineered donor cells (which may also be called feeder cells) can be co-cultured in vitro for different times and at different ratios in order to identify optimal conditions for TIMIT. In some embodiments, co-culture is performed for about 5 minutes to about 2 hours (e.g., about 5, 10, 15, 20, 25, 30, 35, 40, 45, 50, 55, 60, 65, 70, 75, 80, 85, 90, 95, 100, 105, 110, 115, or 120 minutes). Longer and short co-culture times may be appropriate based on culture variables (e.g., temperature, medium, etc.).

In some embodiments in vitro, it may be advantageous to separate donor and acceptor cells using, for instance, a semi-permeable membrane or other membrane that can permit trogocytosis to occur but without mixing of the donor and acceptor cell populations. Suitable transwell systems are readily known and available in the art for tissue culturing in vitro.

Acceptor or target cells can be essentially any cell type if it is capable of receiving or taking up membrane material from a donor cell via trogocytosis. Thus, in some embodiments, the target cell comprises a molecule that is capable of specific interaction with a molecule on the donor cell that helps facilitate trogocytosis. In some embodiments, the acceptor cells are primary human cells. In other embodiments, the acceptor cells are cells of a cell line, or cells of a non-human species. In another embodiment, the target cells are pluripotent cells. Suitable target cells are described more herein.

In order to deliver RNA using the trogocytosis-based delivery methods of this disclosure, the introduced fusion construct comprises a specific RNA binding protein fused to a transmembrane receptor. In such embodiments, RNAs to be transferred may be engineered to comprise one or more sequences that comprise binding regions for the specific RNA binding protein. The donor cell thus can comprise one or more nucleic acid constructs that endcode the RNA of interest (that contains the binding regions to the RNA binding protein). In some cases, the fusion construct comprises a furin cleavage site-containing linker between the RNA binding protein and transmembrane receptor.

In order to deliver DNA using the trogocytosis-based delivery methods of this disclosure, the introduced fusion construct comprises a specific DNA binding protein fused to a transmembrane receptor. In some cases, the fusion construct comprises a furin cleavage site-containing linker between the DNA binding protein and transmembrane receptor.

In some embodiments, provided herein is a method for delivering a gene editing reagent to a target cell where the method comprises introducing into a donor cell (i) one or more nucleic acids encoding one or more nucleic acids encoding a fusion protein comprising a transmembrane receptor fused to a gene editing nuclease (e.g., a Cas nuclease), (ii) one or more gRNAs having complementarity to a target nucleic acid sequence to be genetically modified in the acceptor cell; and, in some cases, (iii) an exogenous nucleic acid sequence encoding a chimeric antigen receptor (CAR), wherein the CAR comprises a ligand-binding domain having specificity for an antigen present on the target cell, whereby a genetically modified human lymphocyte that expresses the CAR is obtained. The method further comprises co-culturing the genetically modified cell with the target cell under conditions that promote trogocytosis between the genetically modified donor cell and the acceptor cell, thus mediating delivery of the cargo to the acceptor cell.

In some embodiments, the target or acceptor cell is an immune cell (e.g., T cell, B cell, tumor-infiltrating lymphocyte (TIL), dendritic cell) or a human pluripotent stem cell. Other target/acceptor cell types include, without limitation, CD34+ hematopoietic stem cells (HSCs), tumor cells, hepatocytes, liver stellate cells, aortic smooth muscle cells, cardiac myocytes, neural cells (e.g., neurons, macroglia, microglia), microglia, fibroblasts, keratinocytes (e.g., LGR5⁺ keratinocytes), epithelial cells, hair follicle stem cells, and muscle cells and progenitors thereof (e.g., PAX7⁺ muscle stem cells).

In another embodiment, the methods of this disclosure can be used to deliver gene editing reagents to target cells to remove, inactivate, or otherwise reduce the activity of a gene in the target cell. For example, a B cell, T cell or NK cell may act as donor cell capable of delivering the base editors to target cells along with one or more guide RNA to direct the gene editing. In one embodiment, a B cell donor acts to transfer a Cas protein, e.g., Cas9, and a gRNA, e.g., a gRNA or a gRNA associated with an RNA binding protein to a target T cell, e.g., a CD4 T cell. In some cases, an exogenous nucleic acid sequence encoding a chimeric antigen receptor (CAR) is also added to donor cell, wherein the CAR comprises a ligand-binding domain having specificity for an antigen present on the target cell, whereby a genetically modified donor cell (e.g., lymphocyte) that expresses the CAR is obtained. The method further comprises co-culturing the genetically modified cell with the target cell under conditions that promote trogocytosis between the genetically modified donor cell and the acceptor target cell, thus mediating delivery of the cargo (gene editing machinery) to the acceptor target cell.

In another embodiment, the methods of this disclosure can be used to deliver gene editing reagents to target cells to remove, inactivate, or otherwise reduce the virulence or infectivity of a virus in a host cell. For example, a B cell may act as donors that home to HIV reservoirs in the lymph nodes (or other lymphoid organs) to deliver base editors to infected T cells that mutate and inactivate human immunodeficiency virus (HIV). In one embodiment, a B cell donor acts to transfer a Cas protein, e.g., Cas9, and a gRNA, e.g., a gRNA or a gRNA associated with an RNA binding protein to a target T cell, e.g., a CD4 T cell, that is infected with HIV. In another example, donor cells deliver base editors to reservoirs of human papillomavirus (HPV) that mutate or inactivate HPV in target cells. Thus, in some embodiments, provided herein are methods for delivering a gene editing reagent to a virally infected target cell where the method comprises introducing into a donor B cell (i) one or more nucleic acids encoding one or more nucleic acids encoding a fusion protein comprising a transmembrane receptor fused to a gene editing nuclease (e.g., a Cas nuclease), (ii) one or more gRNAs having complementarity to a target nucleic acid sequence to be genetically modified in the acceptor cell, for example, a viral nucleic acid including, but not limited to, nucleic acids of HIV or HPV; and, in some cases, (iii) an exogenous nucleic acid sequence encoding a chimeric antigen receptor (CAR), wherein the CAR comprises a ligand-binding domain having specificity for an antigen present on the target cell, whereby a genetically modified human lymphocyte that expresses the CAR is obtained. The method further comprises co-culturing the genetically modified cell with the target cell under conditions that promote trogocytosis between the genetically modified donor cell and the acceptor cell, thus mediating delivery of the cargo to the acceptor cell.

By way of example, the methods of this disclosure can be used to deliver gene editing reagents to target cells in order to correct an inherited disease. For instance, liver cells can be gene edited to restore synthesis of lysosomal enzyme α-1-iduronidase (IDUA) which is deficient in patients with the genetic disorder mucopolysaccharidosis type I (MPS I). As another example, immune cells such as TIL can be targeted in vivo by the methods of this disclosure to disrupt intracellular checkpoint genes. Skin cells such as LGR5⁺ keratinocytes can be targeted to correct recessive epidermolysis bullosa or Junctional epidermolysis bullosa (JEB). Thus, in these embodiments, the donor cell comprising the fusion protein comprising a gene editing protein (e.g., Cas9), a fusion protein comprising a RNA binding protein, and one or more guide RNA to gene edit or correct the target cell for the gene associated with the condition.

In some embodiments, the method can further comprise one or more steps designed to enhance TIMIT. For instance, one or more steps can be performed modulate (e.g., increase, decrease) membrane fluidity. The steps may comprise contacting the co-cultured donor and acceptor cells with a membrane fluidity modulating compound such as a cyclodextrin, which modifies cholesterol content in cell membranes. In some cases, the modulating step may include altering temperature or other culture variables. In other cases, donor cells are pre-treated with a conditioning agent to increase susceptibility of the donor cells to membrane transfer via TIMIT.

In another aspect, provided herein are methods for using the genetically modified donor cells and/or modified acceptor target cells described herein. For example, genetically modified donor cells and/or modified acceptor target cells obtainable by the methods disclosed herein may be used for subsequent steps such as research, diagnostics, pharmacological or clinical applications known to the person skilled in the art. Depending on the desired outcome, the method may comprise administering genetically modified donor cells and/or modified target cells to a subject. In some cases, the method can comprise administering genetically modified donor cells comprises a fusion construct comprising the cargo biomolecule and, in some cases, a CAR having specificity for a particular antigen expressed on the surface of a target cell of interest, whereby the genetically modified donor cells will deliver membrane material and the cargo molecule(s) by trogocytosis to the target cell. In some cases, genetically modified donor cells and/or modified acceptor/target cells may be used to treat or prevent a disease or condition in a subject.

In some cases, the condition is cancer or a precancerous condition. The cancer may include, for example, bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer, etc. A hematopoietic cancer and/or lymphoid cancer may include, for example, acute myelogenous leukemia (AML), acute lymphoblastic leukemia (ALL), myelodysplastic syndromes (MDS), non-Hodgkin lymphoma (NHL), chronic myelogenous leukemia (CMIL), Hodgkin's disease, and/or multiple myeloma. The cancer may be a metastatic cancer. The precancerous condition can be a preneoplastic lesion.

In some embodiments, the disease or condition is associated with a single nucleotide polymorphism (SNP) or single nucleotide variant, a neurodegenerative disease, an autoimmune disorder, enzymatic disease, among others, e.g., any genetic disorder treatable by genetic correction.

For example, a genetically modified donor cell may be administered to inhibit the growth of a tumor in a subject. In some embodiments, the tumor may include a solid tumor.

In some embodiments, donor cells are genetically modified ex vivo, and the modified donor cells are then returned to the subject as an autologous transplant as part of or in advance of immunotherapy or gene therapy. As used herein, the term “autologous” is meant to refer to any material derived from the same individual to which it is later to be re-introduced into the individual.

In some embodiments, the genetically modified donor cells described herein may be included in a composition for administration to a subject. The composition may include a pharmaceutical composition and further include a pharmaceutically acceptable carrier. A therapeutically effective amount of the pharmaceutical composition comprising the modified cells may be administered.

Any appropriate method can be performed to confirm successful delivery of the cargo protein, DNA, or RNA to the target cell. For instance, when the fusion protein comprises gene editing reagents, sequencing of the genetic locus targeted for gene editing can be sequenced to determine whether editing occurred. Effects of gene editing or modulation by introduction of therapeutic cargo can be determined by any appropriate methods. For example, methods and techniques for assessing the expression and/or levels of cell markers are known in the art.

Antibodies and reagents for detection of such markers are well known in the art, and readily available. Assays and methods for detecting such markers include, but are not limited to, flow cytometry, including intracellular flow cytometry, ELISA, ELISpot, cytometric bead array or other multiplex methods, Western Blot and other immunoaffinity-based methods.

Although constructs encoding human proteins are described herein, those of skill in the art will appreciate that non-human and/or synthetic amino acid sequences can be used in place of human amino acid sequences. It will also be appreciated that amino acid analogs can be inserted or substituted in place of naturally occurring amino acid residues. As used herein, the term “amino acid analog” refers to amino acid-like compounds that are similar in structure and/or overall shape to one or more of the twenty L-amino acids commonly found in naturally occurring proteins.

Amino acid analogs are either naturally occurring or non-naturally occurring (e.g. synthesized). If an amino acid analog is incorporated by substituting natural amino acids, any of the 20 amino acids commonly found in naturally occurring proteins may be replaced. While amino acids can be replaced (substituted) with amino acid analogs, in some cases amino acid analogs are inserted into a protein. For example, a codon encoding an amino acid analog can be inserted into the polynucleotide encoding the protein.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain, and an organic compound, e.g., a compound that can act as a nucleic acid cleavage agent.

Nucleic acids, proteins, and/or other molecules of this disclosure may be purified. As used herein, purified means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

Nucleic acids, proteins, and/or other molecules of this disclosure may be isolated. As used herein, “isolated” means to separate from at least some of the components with which it is usually associated whether it is derived from a naturally occurring source or made synthetically, in whole or in part.

In interpreting this disclosure, all terms should be interpreted in the broadest possible manner consistent with the context. It is understood that certain adaptations of the invention described in this disclosure are a matter of routine optimization for those skilled in the art, and can be implemented without departing from the spirit of the invention, or the scope of the appended claims.

As used herein, the terms “synthetic” and “engineered” are used interchangeably and refer to the aspect of having been manipulated by the hand of man.

So that the compositions and methods provided herein may more readily be understood, certain terms are defined:

As used in this specification and the appended claims, the singular forms “a,” “an,” and “the” include plural references unless the context clearly dictates otherwise. Any reference to “or” herein is intended to encompass “and/or” unless otherwise stated.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

As used herein, “modifying” (“modify”) one or more target nucleic acid sequences refers to changing all or a portion of a (one or more) target nucleic acid sequence and includes the cleavage, introduction (insertion), replacement, and/or deletion (removal) of all or a portion of a target nucleic acid sequence. All or a portion of a target nucleic acid sequence can be completely or partially modified using the methods provided herein. For example, modifying a target nucleic acid sequence includes replacing all or a portion of a target nucleic acid sequence with one or more nucleotides (e.g., an exogenous nucleic acid sequence) or removing or deleting all or a portion (e.g., one or more nucleotides) of a target nucleic acid sequence. Modifying the one or more target nucleic acid sequences also includes introducing or inserting one or more nucleotides (e.g., an exogenous sequence) into (within) one or more target nucleic acid sequences.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Unless otherwise defined, all technical terms used herein have the same meaning as commonly understood by one of ordinary skill in the art to which this invention belongs. As used herein and in the claims, the singular forms “a,” “an,” and “the” include the singular and the plural reference unless the context clearly indicates otherwise. Thus, for example, a reference to “an agent” includes a single agent and a plurality of such agents. Any reference to “or” herein is intended to encompass “and/or” unless otherwise stated.

Various exemplary embodiments of compositions and methods according to this invention are now described in the following non-limiting Examples. The Examples are offered for illustrative purposes only and are not intended to limit the scope of the present invention in any way. Indeed, various modifications in addition to those shown and described herein will become apparent to those skilled in the art from the foregoing description and the following examples and fall within the scope of the appended claims.

### EXAMPLES

### Example 1—Generating Donor (Feeder) Cells for Transfer of Intracellular Molecules Via Intercellular Trogocytosis (TIMIT)

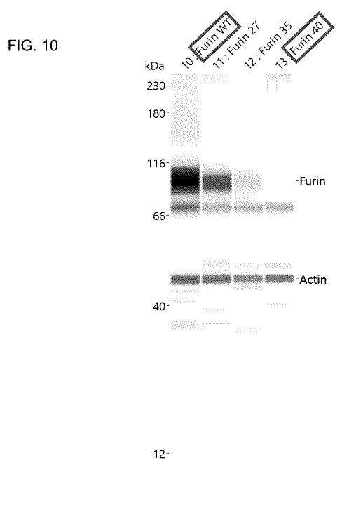

In a first experiment, a furin-linked fusion protein is expressed in a furin knockout donor cell. To this end, CRISPR/Cas9 is used to knockout Furin in K562 cells that express membrane-bound IL21 (mbIL21) and 4-1BBL. Single cell clones are screened by PCR and sequencing of the Cas9 target site in the furin gene, and by western blot analysis for furin protein. Then, a panel of cell surface molecules linked (via a furin cleavage sequence-containing linker sequence) to enhanced GFP (EGFP) on the intracellular side of the cell membrane are assayed. Microscopy is used to assess the amount of EGFP at the inner portion of the cell membrane in furin wild-type and furin-knockout donor cells. As shown in FIG. 10, furin-deficient cell line is produced that does not express furin within the cell.

In another experiment, various trogocytosis delivery methods are tested for delivery of protein, RNA, or DNA to primary human NK cells using furin knockout K562 donor cells that express membrane-bound IL-21 (mbIL-21) with 4-1BB ligand. For protein delivery by trogocytosis, constructs that comprise a transmembrane receptor fused to a protein of interest on the intracellular side of the cell membrane are expressed in donor cells. For RNA delivery by trogocytosis, a specific RNA binding protein such as MS2 is fused to the transmembrane receptor in place of EGFP in the example above. The RNAs to be transferred are engineered to contain one or more MS2 RNA loops for recognition and binding by MS2. For DNA delivery by trogocytosis, a specific DNA binding protein such as TALE is fused to the transmembrane receptor in place of EGFP in the example above. Alternatively, a DNA conjugating enzymes like HUH is used instead of TALE. Engineered donor cells are co-cultured in vitro with acceptor cells under various culture conditions.

For in vivo cargo delivery, engineered donor cells comprising the cargo-containing fusion construct in vivo are administered to humanized immunodeficient mice using the same approaches as described above for in vitro and ex vivo delivery of protein, RNA, or DNA. It may be advantageous in some embodiments to irradiate the engineered donor cells prior to administration so they cannot proliferate and will not persist following administration. Feeder cells are engineered for specific interaction of immune cell types based on ligand and receptor molecules. For instance, K562 feeder cells expressing membrane-bound IL-21 (mbIL-21) with 4-1BB ligand are engineered to interact with NK cells. Since Raji cells do not activate or perform trogocytosis with NK cells, Raji cells are engineered to comprise T cell ligand receptors (i.e., CD3/CD28) to perform in vivo trogocytosis delivery to T cells. In such cases, the donor cells are irradiated as NK92 cells are for use in cancer immunotherapy. The outcome with this approach therefore is in vivo activation, expansion, and engineering of specific immune cell subsets using donor cells and TIMIT.

### Example 2—Trogocytosis NK Co-Culture Assay

In this experiment, Clone9.mbIl21 feeder cells and “Furin KO” (double guide) cells were electroporated with 100 uL Neon. For each 100K cells, 0.5 ug of plasmid was used. Feeder cells were cultured and recovered for about 48 hours prior to co-culture with the KO cells.

Components of novel constructs (amino acid
sequences):
CCR7 (SEQ ID NO: 1):
MDLGKPMKSVLVVALLVIFQVCLCQDEVIDDYIGDNTTVDYTLFESL
CSKKDVRNFKAWFLPIMYSIICFVGLLGNGLVVLTYIYFKRLKTMTD
TYLLNLAVADILFLLTLPFWAYSAAKSWVFGVHFCKLIFAIYKMSFF
SGMLLLLCISIDRYVAIVQAVSAHRHRARVLLISKLSCVGIWILATV
LSIPELLYSDLQRSSSEQAMRCSLITEHVEAFITIQVAQMVIGFLVP
LLAMSFCYLVIIRTLLQARNFERNKAIKVIIAVVVVFIVFQLPYNGV
VLAQTVANFNITSSTCELSKQLNIAYDVTYSLACVRCCVNPFLYAFI
GVKFRNDLFKLFKDLGCLSQEQLRQWSSCRHIRRSSMSVEAETTTTF
SP
XTEN linker (SEQ ID NO: 2)-
GSETPGTSESATPES
FurinSite (SEQ ID NO: 3)-
RRKR
Nuclear localization sequence (NLS, SEQ ID NO: 5)
PKKKRKV
TfnR (TM) (SEQ ID NO: 6)-
MMVDGDNSHVEMKLAVDEEENADNNTKANVTKPKRCSGSICYGTIAV
IVFFLIGFMIGYLGYCKSSDGPGETG
Plasmids:
pRRL MND CCR7-XTEN-FurinSite-GFP
pRRL MND CCR7-XTEN-FurinSite-NLS-GFP
pRRL MND TfnR-XTEN-FurinSite-GFP

Further experiments using the following constructs were used:

CCR7-linker (XTEN)-FurinSite-GFP (SEQ ID NO: 7)
which encodes the fusion protein with sequence
SEQ ID NO: 8:
MDLGKPMKSVLVVALLVIFQVCLCQDEVTDDYIGDNTTVDYTLFESL
CSKKDVRNFKAWFLPIMYSIICFVGLLGNGLVVLTYIYFKRLKTMTD
TYLLNLAVADILFLLTLPFWAYSAAKSWVFGVHFCKLIFAIYKMSFF
SGMLLLLCISIDRYVAIVQAVSAHRHRARVLLISKLSCVGIWILATV
LSIPELLYSDLQRSSSEQAMRCSLITEHVEAFITIQVAQMVIGFLVP
LLAMSFCYLVIIRTLLQARNFERNKAIKVIIAVVVVFIVFQLPYNGV
VLAQTVANFNITSSTCELSKQLNIAYDVTYSLACVRCCVNPFLYAFI
GVKFRNDLFKLFKDLGCLSQEQLRQWSSCRHIRRSSMSVEAETTTTF
SPSGGSSGGSSGSETPGTSESATPESSGGSSGGSRRKRMVSKGEELF
TGVVPILVELDGDVNGHKFSVSGEGEGDATYGKLTLKFICTTGKLPV
PWPTLVTTLTYGVQCFSRYPDHMKQHDFFKSAMPEGYVQERTIFFKD
DGNYKTRAEVKFEGDTLVNRIELKGIDFKEDGNILGHKLEYNYNSHN
VYIMADKQKNGIKVNFKIRHNIEDGSVQLADHYQQNTPIGDGPVLLP
DNHYLSTQSALSKDPNEKRDHMVLLEFVTAAGITLGMDELYK*
CCR7-linker (XTEN)-FurinSite-NLS-GFP (SEQ ID
NO: 9): which encodes the fusion protein with
the sequence SEQ ID NO: 10
MDLGKPMKSVLVVALLVIFQVCLCQDEVIDDYIGDNTTVDYTLFESL
CSKKDVRNFKAWFLPIMYSIICFVGLLGNGLVVLTYIYFKRLKTMTD
TYLLNLAVADILFLLTLPFWAYSAAKSWVFGVHFCKLIFAIYKMSFF
SGMLLLLCISIDRYVAIVQAVSAHRHRARVLLISKLSCVGIWILATV
LSIPELLYSDLQRSSSEQAMRCSLITEHVEAFITIQVAQMVIGFLVP
LLAMSFCYLVIIRTLLQARNFERNKAIKVIIAVVVVFIVFQLPYNGV
VLAQTVANFNITSSTCELSKQLNIAYDVTYSLACVRCCVNPFLYAFI
GVKFRNDLFKLFKDLGCLSQEQLRQWSSCRHIRRSSMSVEAETTTTF
SPSGGSSGGSSGSETPGTSESATPESSGGSSGGSRRKRPKKKRKVSG
GSMVSKGEELFTGVVPILVELDGDVNGHKFSVSGEGEGDATYGKLTL
KFICTTGKLPVPWPTLVTTLTYGVQCFSRYPDHMKQHDFFKSAMPEG
YVQERTIFFKDDGNYKTRAEVKFEGDTLVNRIELKGIDFKEDGNILG
HKLEYNYNSHNVYIMADKQKNGIKVNFKIRHNIEDGSVQLADHYQQN
TPIGDGPVLLPDNHYLSTQSALSKDPNEKRDHMVLLEFVTAAGITLG
MDELYK*
TfnR(TM)-linker (XTEN)-FurinSite-GFP (SEQ ID
NO: 11): which encodes the fusion protein with
sequence SEQ ID NO: 12:
MMVDGDNSHVEMKLAVDEEENADNNTKANVTKPKRCSGSICYGTIAV
IVFFLIGFMIGYLGYCKSSDGPGETGSGGSSGGSSGSETPGTSESAT
PESSGGSSGGSRRKRMVSKGEELFTGVVPILVELDGDVNGHKFSVSG
EGEGDATYGKLTLKFICTTGKLPVPWPTLVTTLTYGVQCFSRYPDHM
KQHDFFKSAMPEGYVQERTIFFKDDGNYKTRAEVKFEGDTLVNRIEL
KGIDFKEDGNILGHKLEYNYNSHNVYIMADKQKNGIKVNFKIRHNIE
DGSVQLADHYQQNTPIGDGPVLLPDNHYLSTQSALSKDPNEKRDHMV
LLEFVTAAGITLGMDELYK*
TfnR(TM)-linker (XTEN)-FurinSite-NLS-GFP
(SEQ ID NO: 13), which encodes the fusion
protein with SEQ ID NO: 14:
MMVDGDNSHVEMKLAVDEEENADNNTKANVTKPKRCSGSICYGTIAV
IVFFLIGFMIGYLGYCKSSDGPGETGSGGSSGGSSGSETPGTSESAT
PESSGGSSGGSRRKRPKKKRKVSGGSMVSKGEELFTGVVPILVELDG
DVNGHKFSVSGEGEGDATYGKLTLKFICTTGKLPVPWPTLVTTLTYG
VQCFSRYPDHMKQHDFFKSAMPEGYVQERTIFFKDDGNYKTRAEVKF
EGDTLVNRIELKGIDFKEDGNILGHKLEYNYNSHNVYIMADKQKNGI
KVNFKIRHNIEDGSVQLADHYQQNTPIGDGPVLLPDNHYLSTQSALS
KDPNEKRDHMVLLEFVTAAGITLGMDELYK*

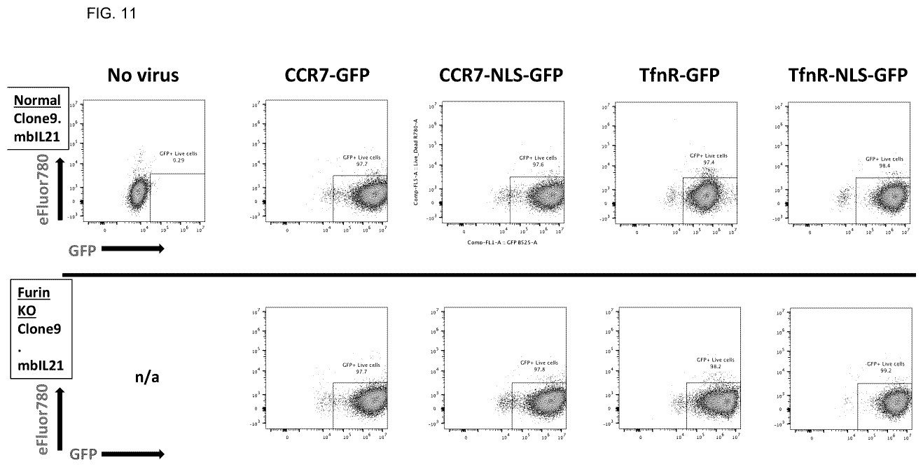

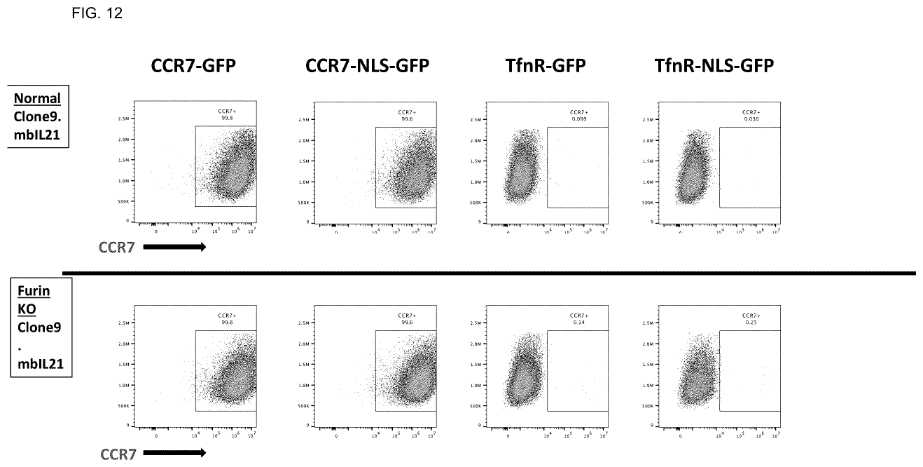

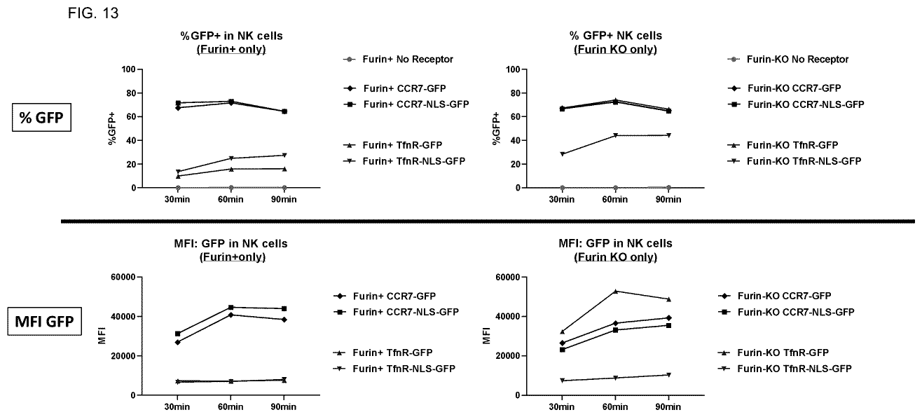

Furin containing cells (Clone9.mbIL21) and Furin KO cells (Clone9.mbIL21 Furin KO Clone40) were transduced at 24 hr in viral supernatant, 24 hr in fresh viral supernatant+F108 (GFP expression in Feeders checked at 48 hr total transduction) and 72 hr in fresh viral supernatant+F108 (GFP expression in Feeders checked at 5 days total transduction). GFP expression by the cells was then detected for the cells containing the different constructs, as demonstrated in FIG. 11 at 11 days total transduction. Cells were measured via flow cytometry and gated for GFP expression and viability. Next, we checked for expression of CCR7 in the furin containing and furin KO cells containing the CCR7-GFP or TfnR-GFP constructs at 11 days post transfection, as demonstrated in FIG. 12.

Co-Culture Set-up: Feeder cells (Normal or Furin-KO) expressing CCR7 or TfnR™ constructs, as well as normal Clone9.mbIL21 feeders (no CCR7/TfnR/GFP expression), were stained with CellTrace Violet dye, then co-cultured with NK cells at 1:1 ratio: for 2 hours, 1 hour, 30 minutes, or 0 hours (“0 hr”). For “0 hr,” Feeders and NK cells were stained separately, then combined immediately preceding data acquisition. In this manner, each cell population could be identified using a flow cytometer. “Feeder only” and “NK only” controls were also collected. At appropriate time points, co-cultures were stained using viability dye, then surface antibody stains.

### Final Panel:

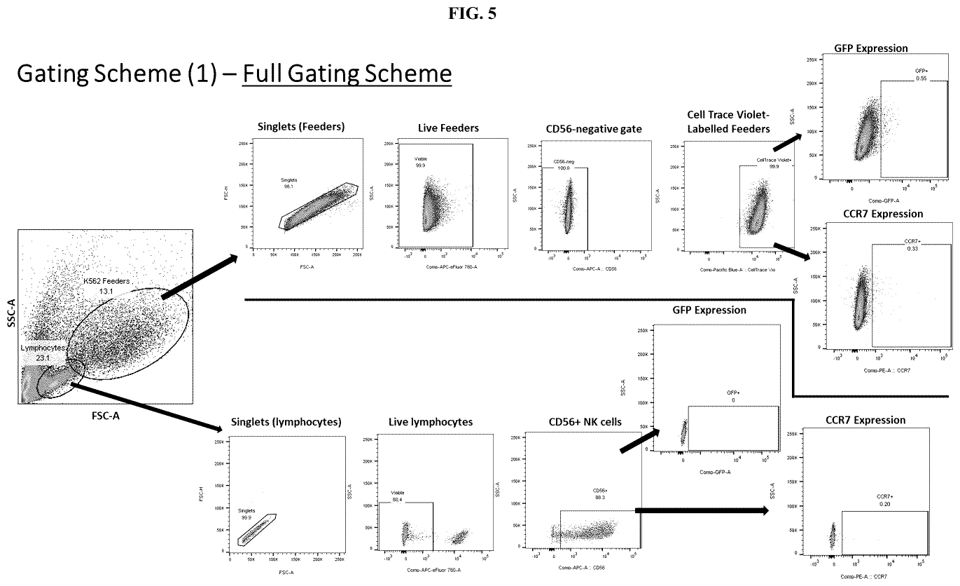

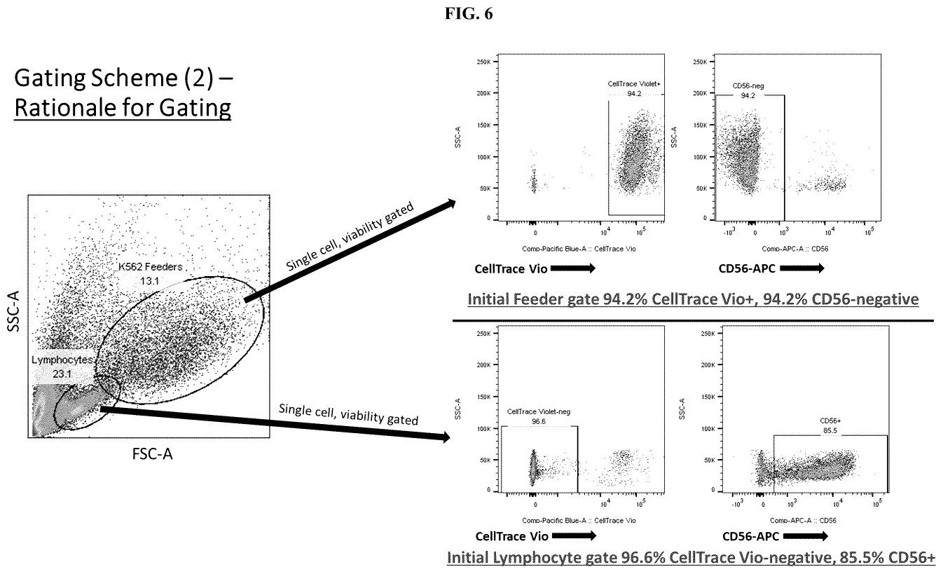

CellTrace Violet-Feeders//CD56-APC-NK cells//GFP//CCR7-PE//Fixable viability dye eFluor780
   FIG. 5 presents the full gating scheme, in which K562 feeder GFP & CCR7 gating was determined using fully-stained non-electroporated Clone9.mbIL21 (which do not express GFP/CCR7). NK GFP & CCR7 gating was determined using fully-stained, non-co-cultured WT NK Cells. FIG. 6 demonstrates the rationale for gating. Single cell viability gating was performed. The initial Feeder gate was 94.2% CellTrace Vio+, 94.2% CD56-negative. The initial Lymphocyte gate 96.6% CellTrace Vio-negative, 85.5% CD56+.

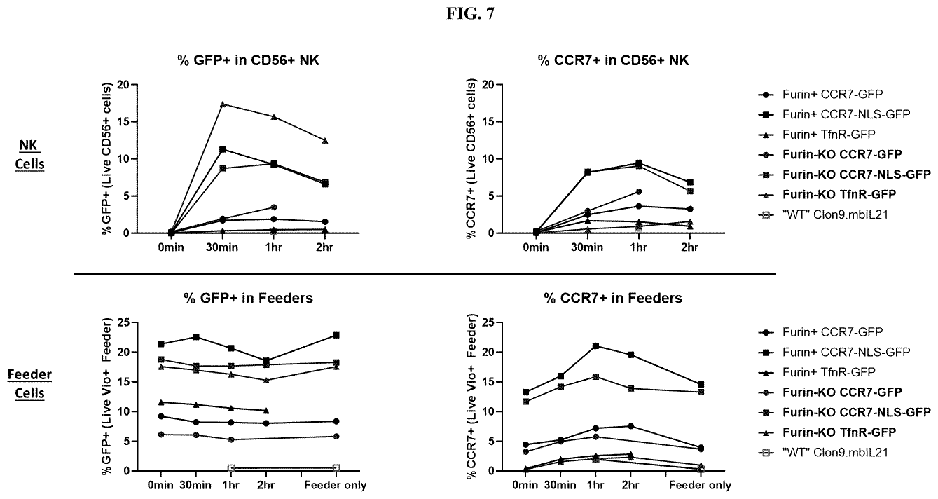

FIG. 7 presents the percentage of GFP+ cells and percentage of CCR7+ cells following co-culture of NK cells and feeder cells. It is noted that NK cells alone, or in co-culture with “WT” Feeders, show ˜0% GFP and CCR7 expression. In general, initial expression level of GFP/CCR7 in Feeder correlated to expression in NK cells following co-culture. GFP and CCR7 expression was constant over the duration of co-culture in feeder cells. In the lower right-hand panel of FIG. 7, the data demonstrate background CCR7 staining in TfnR-GFP or “WT” Feeder samples.

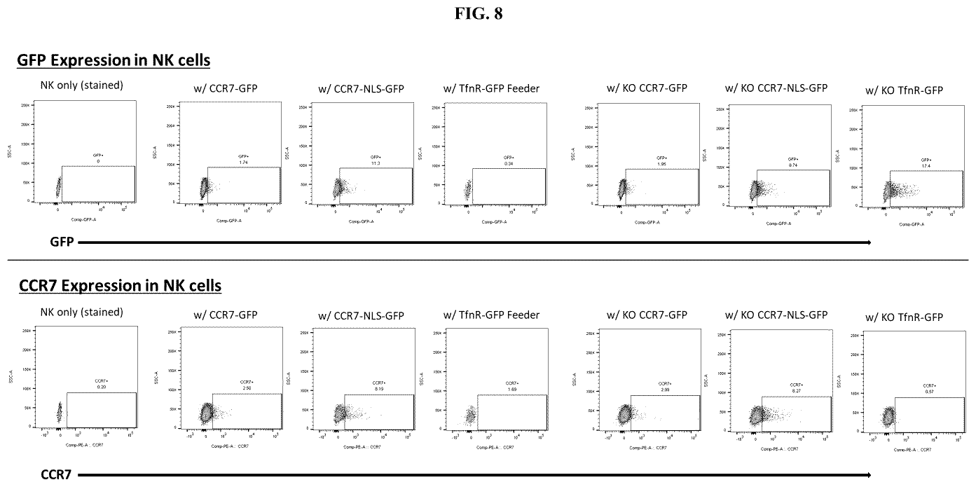

FIG. 8 demonstrates representative GFP and CCR7 Expression in NK cells following about 30 minutes of co-culture, gated on CD56+NK Cells.

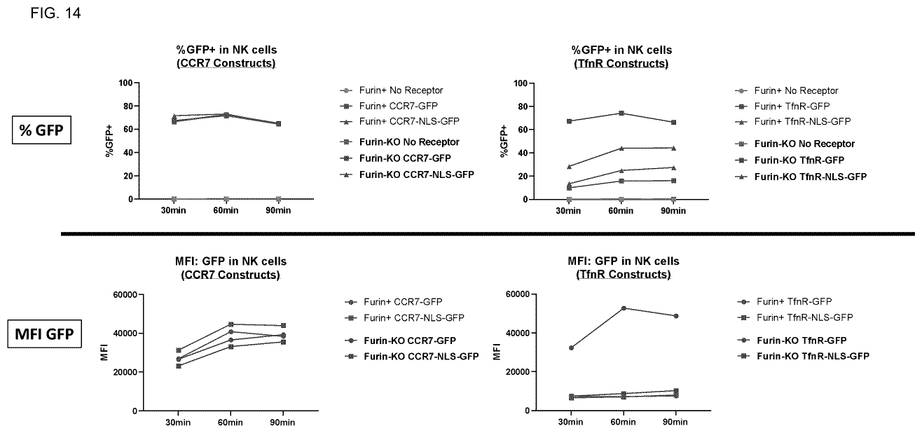

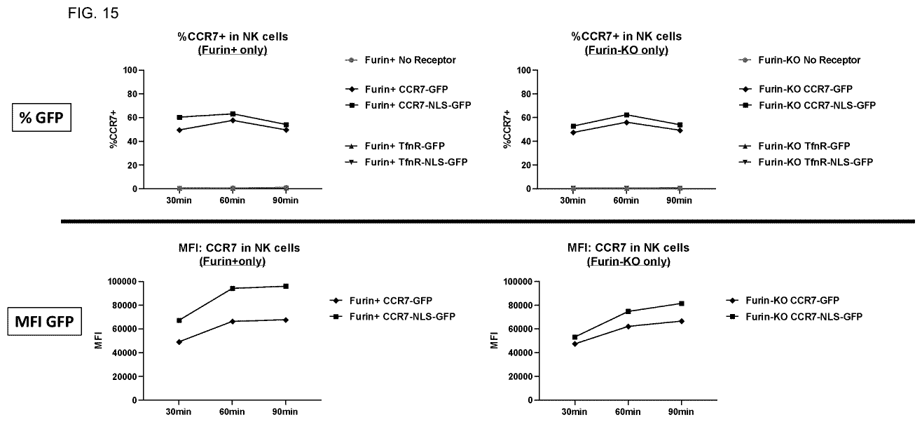

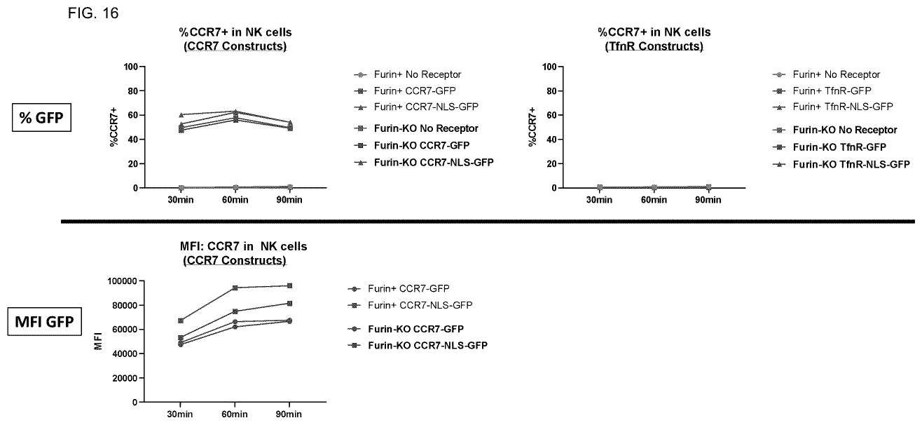

Additional experiments were run at 30 minutes, 60 minutes, and 90 minutes time points and GFP expression was determined, as demonstrated in FIG. 13-16. As shown in FIG. 13, transfer of GFP was much more efficient using the CCR7 construct. FIG. 14 demonstrates the GFP expression in NK cells in the Furin+vs Furin-KO feeder cells as both percent GFP positive and as mean fluorescent intensity (MFI). FIGS. 15 and 16 demonstrates CCR7 expression in NK cells incubated with the feeder cells (furin+ and furin-KO). Overall, the data shows a fusion protein construct of interest may be expressed by a donor cell and transferred to a target cell of interest regardless of the size of an extracellular domain or whether the construct is expressed solely in the nucleus of the donor cell.

### Example 3: Feeder Cell Line Capable of Specific mRNA Transfer

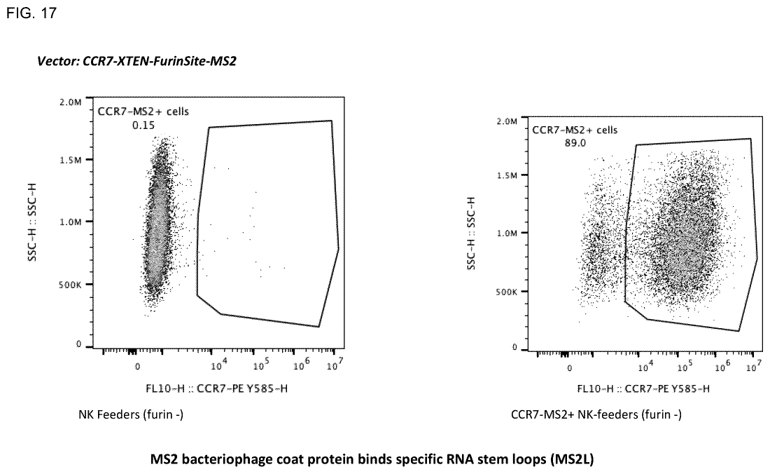

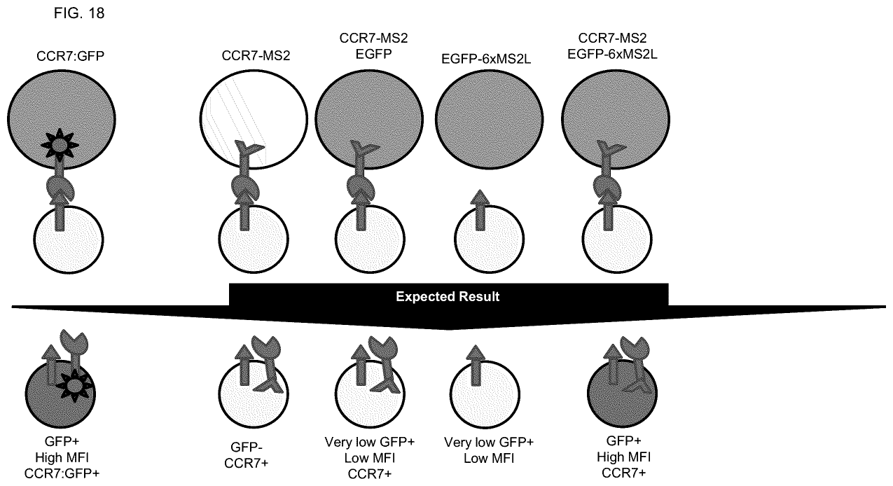

The methods above were used to generate feeder cell like that can facilitate transfer of specific mRNA. A vector construct was made containing a fusion protein of CCR7 (SEQ ID NO:1)-linker (e.g., XTEN, SEQ ID NO:2)—and optional FurinSite (SEQ ID NO:3) and one or more copies of MS2L (SEQ ID NO: 15). Cells expressing the MS2 bacteriophage coat protein were created (FIG. 17) and capable of binding specific RNA stem loops (MS2L). FIG. 18 shows the expected outcomes for the different feeder cell types, demonstrating that it is only expected to see the biomolecule of interest (demonstrated as green) will be transferred most efficiently by feeder cells expressing the CCR7 and the MS2 or biomolecule of interest to the NK cells. This method thus allows for different biomolecules to be delivered (e.g., Crispr and guide RNAs) to target NK cells to initiate downstream events.

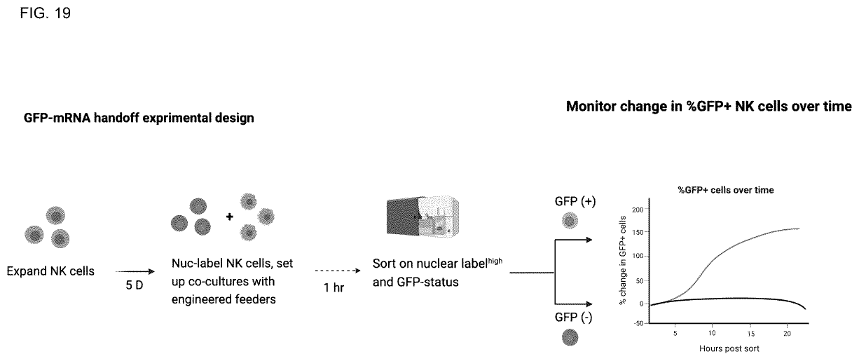

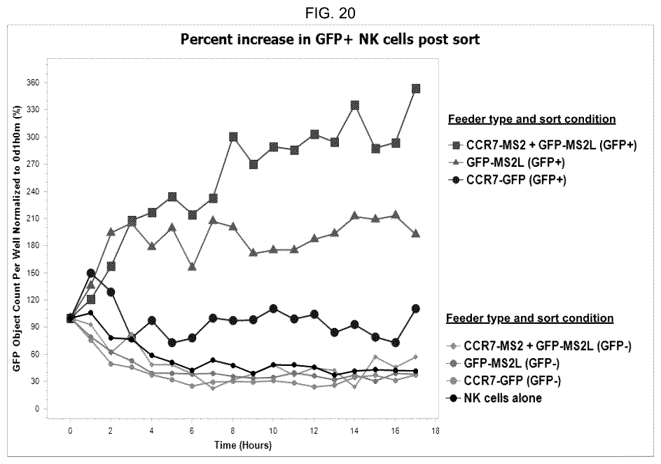

To demonstrate the specificity of the methods described herein, GFP=mRNA handoff experiment was designed, as depicted in FIG. 19. Specifically, NK T cells are incubated with engineered feeder cells that carry GFP mRNA. The cells are co-cultured for an hour, sorted for NK cells having GFP positivity, and the NK cells are monitored over time. As depicted in FIG. 20, GFP positive NK cells post sort show an increase in number of GFP-expressing NK cells.

We claim:

1. A method for delivering a cargo molecule to a target cell, the method comprising:
(a) introducing one or more nucleic acids encoding a fusion protein comprising a transmembrane receptor and the cargo molecule into a donor cell producing a modified donor cell expressing the fusion protein; and
(b) co-culturing the modified donor cell with the target cell, whereby a modified target cell comprising the fusion protein of the modified donor cell is obtained.

2. The method of claim 1, wherein the target cell is a cell that expresses a molecule that specifically binds to expressed molecule on the surface of the donor cell.

3. The method of any one of the preceding claims, wherein the fusion protein further comprises a linker between the transmembrane receptor and cargo molecule, optionally wherein the linker comprises one or more furin cleavage sites.

4. The method any one of the preceding claims, wherein the donor cell is a human monocyte, macrophage, natural killer (NK) cell, cytotoxic T cell, regulatory T cell, B cell, or gamma-delta T cell.

5. The method any one of the preceding claims, wherein the target cell is a human cell.

6. The method of claim 5, wherein the human cell is a T cell, B cell, CD34+ hematopoietic stem cell (HSC), natural killer cell, a tumor cell, hepatocyte, liver stellate cell, neuron, microglia, fibroblast, keratinocyte, epithelial cell, hair follicle stem cell, or muscle cell or a progenitor thereof.

7. The method of any one of the preceding claims, wherein the cargo molecule is a RNA binding protein capable of binding one or more cargo RNA molecules.

8. The method of claim 7, wherein the fusion protein comprises two or more copies of the RNA binding molecule.

9. The method of claim 7 or 8, wherein the one or more RNA binding molecule comprises the RNA binding portion of the bacteriophage M2 protein.

10. The method of any one of claims 1-6, wherein the cargo molecule is a DNA binding protein bound to a cargo DNA molecule of interest.

11. The method of claim 10, wherein the DNA binding protein is a Transcription activator-like effector (TALE) or a HUH endonuclease, or a portion thereof.

12. The method of any one of the preceding claims, wherein the donor cell lacks expression of furin.

13. The method of any one of the preceding claims, wherein the fusion protein comprises one or more furin cleavage sites between the transmembrane receptor and cargo molecule, and wherein the modified target cell comprises endogenous furin capable of cleaving the fusion protein at the one or more furin cleavage sites to release the cargo molecule.

14. The method of claim 10 or 11, wherein the method further comprises prior to step (a),
genetically modifying the donor cell to lack expression or reduce expression of furin.

15. The method of claim 12, wherein the donor cell is genetically modified by gene editing to lack furin.

16. The method of any one of the preceding claims, wherein step (a) comprises the introducing the one or more nucleic acids into the furin locus of the donor cell thereby reducing or preventing expression of endogenous furin in the donor cell.

17. The method of any one of the preceding claims, wherein the cargo molecule is a protein.

18. The method of claim 17, wherein the protein is a gene editing reagent.

19. The method of claim 18, wherein the gene editing reagent is selected from a Cas nuclease, zinc finger nuclease (ZFN), TALEN, base editor, prime editor, transposon/transposase, and integrase.

20. The method of any one of the proceeding claims, wherein the method is performed in vivo, in vitro, or ex vivo.

21. The method of any one of the preceding claims, wherein the transmembrane receptor is a CCR7 protein or binding fragment thereof.

22. The method of any one of the preceding claims, wherein the cargo molecule is a gene editing agent.

23. The method of any one of the preceding claims, wherein the fusion protein comprises CCR7 and a gene editing reagent.

24. A modified target cell obtained according to the method of any one of claims 1-23.

25. Use of the modified target cell of claim 24 for treating a disease in a subject in need thereof.

26. The use of claim 25, wherein the disease is cancer, autoimmune disease, or enzymatic disorder.

27. Method of treating a subject having a disease, the method comprising administering the target cell of claim 24 in an amount effective to treat the disease.

28. The method of claim 27, wherein the subject has a disease selected from cancer, autoimmune disease, or enzymatic disorder.

29. A genetically modified human donor cell comprising one or more nucleic acids encoding a fusion protein comprising a transmembrane receptor and a cargo molecule.

30. The genetically modified donor cell of claim 29, wherein the fusion protein further comprises a linker, one or more furin cleavage sites between the transmembrane receptor and cargo molecule, or combination of both.

31. The genetically modified donor cell of claim 29 or 30, wherein the donor cell is deficient in expression of furin.

32. The genetically modified human donor cell of any one of claims 29-31, wherein the human donor cell is a human monocyte, macrophage, natural killer (NK) cell, cytotoxic T cell, regulatory T cell, or gamma-delta T cell.

33. The genetically modified human donor cell of any one of claim 29-32, wherein the fusion protein is introduced to the donor cell genome at the furin locus, whereby the modified donor cell does not express endogenous furin.

34. The genetically modified human donor cell of any one of claims 29-33, wherein the human donor cell is further genetically modified to express a chimeric antigen receptor (CAR) comprising a single-chain variant fragment (scFv) specific for a target cell, whereby said genetically-modified human cell expresses said CAR.

35. The genetically modified human donor cell of claim 34, wherein the ligand-binding domain of the CAR is specific for CD34 and wherein the target cell is a CD34⁺ hematopoietic stem cell (HSC).

36. A method of treating a subject with a disease, the method comprising administering the genetically modified human donor cell of any one of claims 29-35 in an amount effective to treat the disease.

37. A method for delivering one or more cargo molecule to a target cell for gene editing the target cell, the method comprising:
(a) introducing one or more nucleic acids encoding (a) one or more fusion proteins into a donor cell, wherein each fusion protein comprises a transmembrane receptor and a cargo molecule comprising a gene editing protein, and (ii) one or more guide RNA or DNA encoding a guide RNA, producing a modified donor cell expressing the one or more fusion protein and one or more guide RNAs; and
(b) co-culturing the modified donor cell with the target cell, whereby a modified target cell comprising the fusion protein and guide RNAs of the modified donor cell is obtained and the gene editing protein is capable of editing the target cell.

38. The method of claim 37, wherein the target cell is a cell that expresses a molecule expressed on the surface of the donor cell.

39. The method of any one of the preceding claims, wherein the fusion protein further comprises a linker between the transmembrane receptor and cargo molecule, optionally wherein the linker comprises one or more furin cleavage sites.

40. The method of any one of claims 37-39, wherein the donor cell is a human monocyte, macrophage, natural killer (NK) cell, cytotoxic T cell, regulatory T cell, B cell, or gamma-delta T cell.

41. The method any one of claims 27-40, wherein the target cell is a T cell, B cell, CD34+ hematopoietic stem cell (HSC), natural killer cell, a tumor cell, hepatocyte, liver stellate cell, neuron, microglia, fibroblast, keratinocyte, epithelial cell, hair follicle stem cell, or muscle cell or a progenitor thereof.

42. The method of any one of claims 37-41, wherein the donor cell lacks expression of furin.

43. The method of any one of claims 37-42, wherein the fusion protein comprises one or more furin cleavage sites between the transmembrane receptor and cargo molecule, and wherein the modified target cell comprises endogenous furin capable of cleaving the fusion protein at the one or more furin cleavage sites to release the cargo molecule.

44. The method of claim 42 or 43, wherein the method further comprises prior to step (a),
genetically modifying the donor cell to lack expression or reduce expression of furin.

45. The method of claim 44, wherein the donor cell is genetically modified by gene editing to lack furin.

46. The method of any one of claims 37-45, wherein step (a) comprises the introducing the one or more nucleic acids into the furin locus of the donor cell thereby reducing or preventing expression of endogenous furin in the donor cell.

47. The method of any one of claims 37-46, wherein the gene editing reagent is selected from a Cas nuclease, zinc finger nuclease (ZFN), TALEN, base editor, prime editor, transposon/transposase, and integrase.

48. The method of any one of the proceeding c any one of claims 37-47, wherein the method is performed in vivo, in vitro, or ex vivo.

49. The method of any one of claims 37-48, wherein the transmembrane receptor is a CCR7 protein or binding fragment thereof.

50. The method of any one of claims 37-49, wherein the fusion protein comprises CCR7 and a gene editing reagent.


SEQUENCE LISTING (17 sequences)

SEQ ID NO: 1 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 2 - protein, 15 aa, Artificial Sequence
  Synthetic- XTEN linker
GSETPGTSESATPES

SEQ ID NO: 3 - protein, 4 aa, Artificial Sequence
  Synthetic- FurinSite
RRKR

SEQ ID NO: 4 - protein, 239 aa, Artificial Sequence
  Synthetic- GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - protein, 7 aa, Artificial Sequence
  Synthetic- NLS
PKKKRKV

SEQ ID NO: 6 - protein, 73 aa, Artificial Sequence
  Synthetic- TfnR (TM)
MMVDGDNSHVEMKLAVDEEENADNNTKANVTKPKRCSGSICYGTIAVIVFFLIGFMIGYLGYCKSSDGPGETG

SEQ ID NO: 7 - DNA, 1962 bp, Artificial Sequence
  Synthetic- CCR7-XTEN-FurinSite-GFP
atggacctgggcaagcccatgaagtccgtgctggtggtggccctgctggtcatctttcaggtgtgcctgtgccaggatgaggtgacagacgattacatcggcgacaataccacagtggattataccctgttcgagtctctgtgcagcaagaaggacgtgcggaacttcaaggcctggtttctgcctatcatgtacagcatcatctgtttcgtgggcctgctgggcaatggcctggtggtgctgacatacatctacttcaagcggctgaagaccatgacagacacctacctgctgaacctggccgtggccgatatcctgttcctgctgaccctgccattttgggcctattctgccgccaagagctgggtgttcggcgtgcacttttgcaagctgatctttgccatctacaagatgtccttcttttctggcatgctgctgctgctgtgcatcagcatcgacagatatgtggcaatcgtgcaggccgtgtccgcccaccggcacagagccagggtgctgctgatcagcaagctgtcctgcgtgggcatctggatcctggccaccgtgctgtccatccctgagctgctgtactctgatctgcagagaagctcctctgagcaggccatgaggtgttccctgatcacagagcacgtggaggccttcatcaccatccaggtggcccagatggtcatcggcttcctggtgccactgctggccatgagcttttgctacctggtcatcatcagaaccctgctgcaggcccgcaactttgagcggaataaggccatcaaagtgatcatcgccgtggtggtggtgttcatcgtgtttcagctgccctataatggcgtggtgctggcccagacagtggccaacttcaatatcacaagctccacctgcgagctgtctaagcagctgaacatcgcctacgacgtgacctatagcctggcatgcgtgcggtgctgcgtgaaccccttcctgtatgcctttatcggcgtgaagtttcgcaatgacctgttcaagctgtttaaggatctgggatgcctgagccaggagcagctgaggcagtggtctagctgtcggcacatccggagatcctctatgtccgtggaggccgagaccacaaccacattctctcctagcggaggaagctccggaggatctagcggatctgagacaccaggcacctccgagtctgccacaccagagtcctctggcggcagctccggaggaagcaggcgcaagagaatggtgtccaagggagaggagctgttcaccggagtggtgcctatcctggtggagctggacggcgatgtgaatggccacaagtttagcgtgtccggagagggagagggcgatgcaacatacggcaagctgaccctgaagttcatctgcaccacaggcaagctgcccgtgccttggccaacactggtgaccacactgacctacggcgtgcagtgtttctccaggtatcctgaccacatgaagcagcacgatttctttaagtctgccatgccagagggctacgtgcaggagcgcaccatcttctttaaggacgatggcaactataagacacgggccgaggtgaagttcgagggcgacaccctggtgaacagaatcgagctgaagggcatcgactttaaggaggatggcaatatcctgggccacaagctggagtacaactataattcccacaacgtgtacatcatggccgataagcagaagaacggcatcaaggtgaacttcaagatcaggcacaatatcgaggacggcagcgtgcagctggccgatcactaccagcagaacacaccaatcggcgacggacccgtgctgctgcctgataatcactatctgtctacccagagcgccctgtccaaggaccccaacgagaagcgcgatcacatggtgctgctggagttcgtgacagcagcaggaatcaccctgggaatggacgagctgtataagtga

SEQ ID NO: 8 - protein, 653 aa, Artificial Sequence
  Synthetic- CCR7-XTEN-FurinSite-GFP
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSPSGGSSGGSSGSETPGTSESATPESSGGSSGGSRRKRMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 9 - DNA, 1995 bp, Artificial Sequence
  Synthetic- CCR7-XTEN-FurinSite-NLS-GFP
atggacctgggcaagcccatgaagtccgtgctggtggtggccctgctggtcatctttcaggtgtgcctgtgccaggatgaggtgacagacgattacatcggcgacaataccacagtggattataccctgttcgagtctctgtgcagcaagaaggacgtgcggaacttcaaggcctggtttctgcctatcatgtacagcatcatctgtttcgtgggcctgctgggcaatggcctggtggtgctgacatacatctacttcaagcggctgaagaccatgacagacacctacctgctgaacctggccgtggccgatatcctgttcctgctgaccctgccattttgggcctattctgccgccaagagctgggtgttcggcgtgcacttttgcaagctgatctttgccatctacaagatgtccttcttttctggcatgctgctgctgctgtgcatcagcatcgacagatatgtggcaatcgtgcaggccgtgtccgcccaccggcacagagccagggtgctgctgatcagcaagctgtcctgcgtgggcatctggatcctggccaccgtgctgtccatccctgagctgctgtactctgatctgcagagaagctcctctgagcaggccatgaggtgttccctgatcacagagcacgtggaggccttcatcaccatccaggtggcccagatggtcatcggcttcctggtgccactgctggccatgagcttttgctacctggtcatcatcagaaccctgctgcaggcccgcaactttgagcggaataaggccatcaaagtgatcatcgccgtggtggtggtgttcatcgtgtttcagctgccctataatggcgtggtgctggcccagacagtggccaacttcaatatcacaagctccacctgcgagctgtctaagcagctgaacatcgcctacgacgtgacctatagcctggcatgcgtgcggtgctgcgtgaaccccttcctgtatgcctttatcggcgtgaagtttcgcaatgacctgttcaagctgtttaaggatctgggatgcctgagccaggagcagctgaggcagtggtctagctgtcggcacatccggagatcctctatgtccgtggaggccgagaccacaaccacattctctcctagcggaggaagctccggaggatctagcggatctgagacaccaggcacctccgagtctgccacaccagagtcctctggcggcagctccggaggaagcaggcgcaagagacccaagaagaagcggaaggtgtctggcggcagcatggtgtccaagggagaggagctgttcaccggagtggtgcctatcctggtggagctggacggcgatgtgaatggccacaagtttagcgtgtccggagagggagagggcgatgcaacatacggcaagctgaccctgaagttcatctgcaccacaggcaagctgcccgtgccttggccaacactggtgaccacactgacctacggcgtgcagtgtttctccaggtatcctgaccacatgaagcagcacgatttctttaagtctgccatgccagagggctacgtgcaggagcgcaccatcttctttaaggacgatggcaactataagacacgggccgaggtgaagttcgagggcgacaccctggtgaacagaatcgagctgaagggcatcgactttaaggaggatggcaatatcctgggccacaagctggagtacaactataattcccacaacgtgtacatcatggccgataagcagaagaacggcatcaaggtgaacttcaagatcaggcacaatatcgaggacggcagcgtgcagctggccgatcactaccagcagaacacaccaatcggcgacggacccgtgctgctgcctgataatcactatctgtctacccagagcgccctgtccaaggaccccaacgagaagcgcgatcacatggtgctgctggagttcgtgacagcagcaggaatcaccctgggaatggacgagctgtataagtga

SEQ ID NO: 10 - protein, 664 aa, Artificial Sequence
  Synthetic- CCR7-XTEN-FurinSite-NLS-GFP
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSPSGGSSGGSSGSETPGTSESATPESSGGSSGGSRRKRPKKKRKVSGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 11 - DNA, 1047 bp, Artificial Sequence
  Synthetic- TfnR-XTEN-FurinSite-GFP
atgatggtggacggcgataatagccacgtggagatgaagctggccgtggacgaggaggagaacgccgataacaataccaaggccaatgtgacaaagcctaagcggtgctccggctctatctgttacggcaccatcgccgtgatcgtgttctttctgatcggctttatgatcggctacctgggctattgcaagagctccgacggaccaggagagaccggatccggaggctctagcggaggatcctctggatctgagaccccaggcacaagcgagtccgccacaccagagagctccggaggatctagcggaggatcccggagaaagaggatggtgagcaagggcgaggagctgttcacaggcgtggtgcctatcctggtggagctggacggcgatgtgaatggccacaagttttctgtgagcggagagggagagggcgatgcaacctatggcaagctgacactgaagttcatctgcaccacaggcaagctgcccgtgccttggccaaccctggtgaccacactgacatacggcgtgcagtgtttcagcagatatcctgaccacatgaagcagcacgatttctttaagtccgccatgccagagggctacgtgcaggagcggaccatcttctttaaggacgatggcaactataagaccagagccgaggtgaagttcgagggcgacacactggtgaacaggatcgagctgaagggcatcgactttaaggaggatggcaatatcctgggccacaagctggagtacaactataattctcacaacgtgtacatcatggccgataagcagaagaacggcatcaaggtgaacttcaagatccgccacaatatcgaggacggctccgtgcagctggccgatcactaccagcagaacaccccaatcggcgacggacccgtgctgctgcctgataatcactatctgtccacacagtctgccctgagcaaggaccccaacgagaagcgggatcacatggtgctgctggagttcgtgaccgcagcaggaatcacactgggaatggacgagctgtataagtga

SEQ ID NO: 12 - protein, 348 aa, Artificial Sequence
  Synthetic- TfnR-XTEN-FurinSite-GFP
MMVDGDNSHVEMKLAVDEEENADNNTKANVTKPKRCSGSICYGTIAVIVFFLIGFMIGYLGYCKSSDGPGETGSGGSSGGSSGSETPGTSESATPESSGGSSGGSRRKRMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 13 - DNA, 1080 bp, Artificial Sequence
  Synthetic- TfnR(TM)-linker (XTEN)-FurinSite-NLS-GFP
atgatggtggacggcgataatagccacgtggagatgaagctggccgtggacgaggaggagaacgccgataacaataccaaggccaatgtgacaaagcctaagcggtgctccggctctatctgttacggcaccatcgccgtgatcgtgttctttctgatcggctttatgatcggctacctgggctattgcaagagctccgacggaccaggagagaccggatccggaggctctagcggaggatcctctggatctgagaccccaggcacaagcgagtccgccacaccagagagctccggaggatctagcggaggatcccggagaaagaggcccaagaagaagcggaaggtgtccggcggctctatggtgagcaagggcgaggagctgttcacaggcgtggtgcctatcctggtggagctggacggcgatgtgaatggccacaagttttctgtgagcggagagggagagggcgatgcaacctatggcaagctgacactgaagttcatctgcaccacaggcaagctgcccgtgccttggccaaccctggtgaccacactgacatacggcgtgcagtgtttcagcagatatcctgaccacatgaagcagcacgatttctttaagtccgccatgccagagggctacgtgcaggagcggaccatcttctttaaggacgatggcaactataagaccagagccgaggtgaagttcgagggcgacacactggtgaacaggatcgagctgaagggcatcgactttaaggaggatggcaatatcctgggccacaagctggagtacaactataattctcacaacgtgtacatcatggccgataagcagaagaacggcatcaaggtgaacttcaagatccgccacaatatcgaggacggctccgtgcagctggccgatcactaccagcagaacaccccaatcggcgacggacccgtgctgctgcctgataatcactatctgtccacacagtctgccctgagcaaggaccccaacgagaagcgggatcacatggtgctgctggagttcgtgaccgcagcaggaatcacactgggaatggacgagctgtataagtga

SEQ ID NO: 14 - protein, 359 aa, Artificial Sequence
  Synthetic- TfnR(TM)-linker (XTEN)-FurinSite-NLS-GFP
MMVDGDNSHVEMKLAVDEEENADNNTKANVTKPKRCSGSICYGTIAVIVFFLIGFMIGYLGYCKSSDGPGETGSGGSSGGSSGSETPGTSESATPESSGGSSGGSRRKRPKKKRKVSGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - DNA, 19 bp, Artificial Sequence
  Synthetic- MS2 loop
acatgaggatcacccatgt

SEQ ID NO: 16 - protein, 33 aa, Artificial Sequence
  Synthetic- TALE binding motif
LTPEQVVAIASHDGGKQALETVQRLLRVCQAHG

SEQ ID NO: 17 - protein, 111 aa, Artificial Sequence
  Synthetic- HUH protein tag
MAKSGNYSYKRWVFTINNPTFEDYVHVLEFCTLDNCKFAIVGEEKGANGTPHLQGFLNLRSNARAAALEESLGGRAWLSRARGSDEDNEEYCAKESTYLRVGEPVSKGRSS